(19) United States
(12) Patent Application Publication
(10) Pub. No.: US 2013/0202698 A1
(43) Pub. Date: Aug. 8, 2013

(54) L-ORNIDAZOLE FORMULATIONS AND THEIR APPLICATIONS IN TREATMENT OF PARASITIC INFECTIONS

(71) Applicant: Nanjing Sanhome Pharmaceutical Co., Ltd., Nanjing (CN)

(72) Inventors: Yong Wang, Nanjing (CN); Cang Zhang, Nanjing (CN); Xiaoxin Tao, Nanjing (CN)

(73) Assignee: NANJING SANHOME PHARMACEUTICAL CO., LTD., Nanjing (CN)

(21) Appl. No.: 13/831,706

(22) Filed: Mar. 15, 2013

Related U.S. Application Data
(63) Continuation-in-part of application No. 11/909,623, filed on Nov. 5, 2007.

(30) Foreign Application Priority Data
Jul. 8, 2005 (CN) 200510083517.2

(51) Int. Cl. A61K 31/4164

(52) U.S. Cl. CPC A61K 31/4164
USPC 424/465; 514/398; 424/44

(57) ABSTRACT

This invention relates to new methods of treating parasitic infections, such as trichomonas vaginalis infection and cecum amoeba infection, using L-enantiomer enriched ornidazole, in particular enantiomerically pure L-ornidazole, which provides benefits such as higher efficacy and lower toxicity to central nervous system over the existing racemic Ornidazole drug. New methods of synthesizing L- and D-enantiomers of Ornidazole in high purity and enantiomeric excess (ee), new formulations of the enantiomerically enriched L- or D-ornidazole, as well as their preparation processes and methods of use, are also disclosed.

### CROSS REFERENCE TO RELATED APPLICATIONS

This application is a Continuation-in-Part application of U.S. patent application Ser. No. 11/909,623, filed on Nov. 5, 2007, which is a U.S. national stage application of International Patent Application No. PCT/CN2006/001204, filed on Jun. 5, 2006, which claims priority to Chinese Patent Application No. 200510083517.2, filed on Jul. 8, 2005, all of which are incorporated herein by reference in their entireties.

### FIELD OF THE INVENTION

This invention relates to anti-parasitic infection drugs prepared from enantiomerically enriched or substantially enantiomerically pure S-(−)-ornidazole (L-ornidazole), in particular their formulations, formulation processes, methods of preparation, and clinical applications in the treatment of parasitic infections, such as trichomonas vaginalis infection and the cecum amoeba infection, with enhanced or comparable efficacy and markedly lowered toxicity.

### BACKGROUND OF THE INVENTION

(1-(3-Chloro-2-hydroxypropyl)-2-methyl-5-nitroimidazole (“Ornidazole”), a third-generation nitroimidazole derivative, is a potent anti-anaerobic and anti-parasitic agent. It has high potency, short clinical course, good tolerance, and good in vivo distribution. The mechanism of action has been proposed to go through the reduction of the nitro group into an amino group under anaerobic environments, or through the formation of a free radical and its interaction with cellular components, resulting in death of microorganisms. Due to the existence of a chiral center, ornidazole contains two enantiomers: the (S)-(−)-enantiomer (“levo-ornidazole”, “L-ornidazole”, “L-enantiomer”, or the like, which are used interchangeably in this application), and the (R)-(+)-enantiomer (“dextro-ornidazole”, “D-ornidazole”, “D-enantiomer”, or the like, which are used interchangeably in this application).

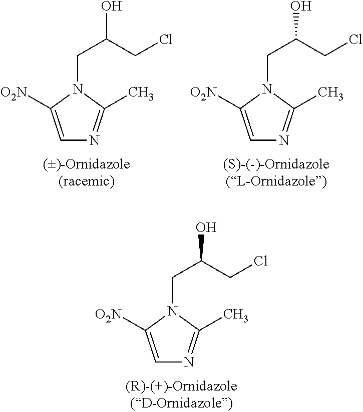

Although ornidazole has been known for over 30 years and used for treatment of parasitic infections, up to the present invention, only its racemic mixture has been used commercially. For example, Ayla Guven (Indian J. Pediatr. 2003; 70(5): p. 437-438) reported use of ornidazole, a racemate, in treating the parasitic infection amebiasis by intravenous administration of ornidazole to a newborn having amebiasis.

While direct comparison of the two enantiomers of ornidazole for possible differences of their anti-parasitic activity and/or toxicity has not been reported, the L- and D-enantiomers have been reported to have same effects on sperm motility, with no significant differences in their spermatozoa and general toxicity. See Bone, W., et al., Int. J. Andrology, 20:347-355 (1997). Bone et al. reported results from a rather thorough comparison among numerous ornizadole analogs, including the L- and D-enantiomers of ornidazole, on their effects on sperm motility. Bone et al. concluded that the motility-inhibiting efficacy of (R)- and (S)-ornidazole on spermatozoa as well as their general toxicity are substantially the same.

Ornidazole shows very good activity in the treatment of parasitic infections clinically, but it also causes some adverse reactions or events, such as vertigo, dizziness, drowsiness, hypersomnia, stomach discomfort, etc. However, no comparative studies on the efficacy or toxicities/adverse events between the L- and D-isomers of ornidazole, or between either of them and a racemic mixture, have been reported. More recently Lin et al. (CN 1400312A) reported a method for separation of racemic ornidazole into L- and D-enantiomers using enzymatic processes, acknowledging Bone et al.'s observation that the two enantiomers of ornidazole do not have apparent differences in efficacy or side effects.

In spite of Bone et al. and Lin et al., the present inventors studied the two enantiomers of ornidazole more systematically on their anti-parasitic activity and toxicities, since development of a chiral ornidazole drug using only one of the enantiomers as the active ingredient could provide benefits such as higher efficacy and/or better safety profile over the existing racemic ornidazole drug. In particular, adverse events and toxicities, mostly occurring in the central nervous system, have limited the use of racemate ornidazole, and the toxicities of the racemic mixture of ornidazole may prevent long-term use in treating infections. Therefore, there has been a need to design and conduct appropriate research and clinical trials in order to realize the full potential and benefits of ornidazole in treating parasitic infections.

### SUMMARY OF THE INVENTION

The present invention fulfills the foregoing need by providing a single enantiomer, the L-ornidazole, or L-enantiomer-enriched ornidazole, as an anti-parasitic agent based on the surprising discovery of the differences between L- and D-isomers of ornidazole in toxicity profiles, in particular the markedly lower toxicity of L-isomer to the central nervous system (CNS). The invention is a result of comprehensive studies to compare the anti-parasitic activity and toxicity between the two enantiomers, and between either of the single enantiomer and the racemate, including the uniquely-designed animal studies and human clinical trials. By conducting hundreds of different testing and experiments, the applicants have concluded that use of L-ornidazole as anti-parasitic agent provides unprecedented benefits to patients over the corresponding existing racemate drug.

A pure pharmaceutical formulation containing substantially only L-ornidazole will bring answers to the unmet medical needs, allowing long-term use in patients without the higher levels of toxicity, as well as potentially allowing administration at increased dosage levels to remove parasitic infections more efficiently with less risk to the patient's safety. Therefore, introduction of a L-ornidazole based chiral medicine will significantly improve clinical safety in treating parasitic infections.

Thus, in one aspect, the present invention provides a method for treating a parasitic infection, comprising administering to a patient in need of treatment a therapeutically effective amount of ornidazole enriched with L-enantiomer (L-ornidazole), or a pharmaceutically acceptable salt, solvate, or prodrug thereof. Administration of the L-enantiomer enriched ornidazole has a diminished inhibitory effect and toxicity on central nervous system of the patient as compared to administration of the corresponding racemic, or dextro-enantiomer enriched, ornidazole.

In another aspect, the present invention provides a method for treating a parasitic infection in a patient, comprising administering to the patient a pharmaceutical composition comprising a therapeutically effective amount of ornidazole enriched with L-enantiomer (L-ornidazole) and a pharmaceutically acceptable carrier. The pharmaceutical composition comprising a therapeutically effective amount of L-enantiomer enriched ornidazole has a diminished inhibitory effect and toxicity on the central nervous system of the patient as compared to a corresponding composition containing a racemic or dextro-enantiomer enriched ornidazole.

In another aspect, the present invention provides pharmaceutical compositions for treatment of parasitic infections, comprising ornidazole enriched with the L-enantiomer. The pharmaceutical compositions are formulated as pharmaceutical dosage forms suitable for oral, intravenous, intraperitoneal, or intravaginal delivery systems.

In another aspect, the present invention provides methods of preparing enantiomerically pure or substantially pure L-ornidazole and D-ornidazole.

In another aspect, the present invention provides methods of preparing the pharmaceutical compositions and dosage forms comprising enantiomerically pure or substantially pure L-ornidazole for treatment of parasitic infections. The parasitic infections include, but are not limited to, trichomonas vaginalis infection and cecal amoeba infection.

These and other aspects of the present invention will be better appreciated by reference to the following drawings and detailed description.

### BRIEF DESCRIPTION OF THE DRAWINGS

FIG. 1 represents an HPLC chromatogram of an L-ornidazole sample on a chiral column.

FIG. 2 represents an HPLC chromatogram of an L-ornidazole sample on a chiral column wherein the L-ornidazole sample was spiked with 0.5% D-ornidazole.

FIG. 3 represents an HPLC chromatogram of a racemic ornidazole sample on a chiral column.

FIG. 4 shows the glutamate levels in (A) cerebrum and (B) cerebellum of mice treated with L- and D-ornidazole enantiomers, respectively.

FIG. 5 shows the effects of L-ornidazole, D-ornidazole, and racemic ornidazole, respectively, on the body weight of Beagle dogs.

FIG. 6 shows the difference in the number of Purkinje cells in cerebellar slices of Beagle dogs treated with L- and D-ornidazole enantiomers (n=4, Mean±SD), respectively.

FIG. 7 shows the different effects of (A) D-ornidazole and (B) L-ornidazole enantiomers on morphology of Purkinje cells.

### DETAILED DESCRIPTION OF THE INVENTION

The present invention was aiming to develop a more effective, less toxic therapy for parasitic infections through comprehensive studies of pharmacokinetics, pharmacodynamics, toxicology, and general pharmacology, among others, of ornidazole, comparing its L-enantiomer with D-enantiomer and racemic mixture. The studies have demonstrated that L-ornidazole has better pharmacokinetic characteristics and lower toxicity to central nervous system than D-ornidazole and racemic ornidazole components.

The experimental data from the studies demonstrate significant differences between L-ornidazole and D-ornidazole and/or the racemate, which was surprising especially in light of the literature reports, such as Bone et al. and Lin et al. The unexpected superior results include markedly reduced adverse events and toxicities associated with the inhibition of the central nervous system by L-ornidazole in both animal and human studies in comparison with D-ornidazole and racemate counterparts. Based on these results, an ornidazole product enriched with L-enantiomer provides benefits of lowered toxicity in comparison with the corresponding racemate drug.

The results are consistent from neurotransmitter molecular level and cell/tissue level as well as animal models and human clinical trials. The superiority of L-ornidazole is demonstrated at both high dosage levels in animal toxicity studies and regular dosage levels in human clinical trials. The differences of L-ornidazole from dextro-ornidazole or racemate are of statistical and practical/clinical significance, because L-ornidazole represents a highly significant improvement in patient safety and diminished adverse events over dextro- or racemic ornidazole. This new treatment regimen will especially benefit patients needing long-term use, for example, in treatment of infections in chronic Crohn's disease for up to one year of treatment.

Specifically, among others, short term toxicology studies in Beagle dogs showed L-ornidazole had lower central nervous system (CNS) toxicity and better safety profiles than D-ornidazole and racemic ornidazole after two weeks of intravenous injections. Pharmacological tests on mice central nervous system showed that L-ornidazole has lower inhibitory effects than the D- or racemic ornidazole. In contrast, the significant inhibitory effect of D-ornidazole on the CNS has been demonstrated by its strong anti-seizure activity in mice pretreated with seizure-inducing agents and by reduced number of the Purkinjie cells in the cerebellar slices of otherwise Beagle dogs treated with D-ornidazole as compared with those treated with L-ornidazole.

Thus, in one aspect, the present invention provides a method for treating a parasitic infection, comprising administering to a patient in need of treatment a therapeutically effective amount of ornidazole enriched with L-enantiomer (L-ornidazole), or a pharmaceutically acceptable salt, solvate, or prodrug thereof.

In one embodiment of this aspect, the administration of the L-enantiomer enriched ornidazole has a diminished inhibitory effect and toxicity on central nervous system of the patient as compared to administration of the corresponding racemic, or dextro-enantiomer enriched, ornidazole.

In another embodiment of this aspect, the L-enantiomer enriched ornidazole has at least 95.0% ee.

In another embodiment of this aspect, the L-enantiomer enriched ornidazole has at least 98.0% ee.

In another embodiment of this aspect, the L-enantiomer enriched ornidazole has at least 99.0% ee.

In another embodiment of this aspect, the L-enantiomer enriched ornidazole has at least 99.5% ee.

In one embodiment of this aspect, the L-enantiomer enriched ornidazole is substantially enantiomerically pure L-ornidazole.

In one embodiment of this aspect, the L-enantiomer enriched ornidazole is enantiomerically pure L-ornidazole, i.e., with a 100% ee.

In another embodiment of this aspect, the L-enantiomer enriched ornidazole contains only L-ornidazole with an undetectable level of D-ornidazole by chiral HPLC.

In another embodiment of this aspect, the parasitic infection is trichomonas vaginalis infection or cecal amoeba infection.

In another aspect, the present invention provides a method for treating a parasitic infection in a patient, comprising administering to the patient a pharmaceutical composition comprising a therapeutically effective amount of ornidazole enriched with L-enantiomer (L-ornidazole) and a pharmaceutically acceptable carrier.

In one embodiment of this aspect, the pharmaceutical composition has a diminished inhibitory effect and toxicity on the central nervous system of the patient as compared to the corresponding composition containing a racemic or dextro-enantiomer enriched ornidazole.

In one embodiment of this aspect, the L-enantiomer enriched ornidazole in the pharmaceutical composition has at least 95.0% ee.

In another embodiment of this aspect, the L-enantiomer enriched ornidazole in the pharmaceutical composition has at least 98.0% ee.

In another embodiment of this aspect, the L-enantiomer enriched ornidazole in the pharmaceutical composition has at least 99.0% ee.

In another embodiment of this aspect, the L-enantiomer enriched ornidazole in the pharmaceutical composition has at least 99.5% ee.

In one embodiment of this aspect, the L-enantiomer enriched ornidazole in the pharmaceutical composition is substantially enantiomerically pure L-ornidazole.

In another embodiment of this aspect, the L-enantiomer enriched ornidazole contains only L-ornidazole with an undetectable level of D-ornidazole by chiral HPLC.

In another embodiment of this aspect, the L-enantiomer enriched ornidazole is substantially enantiomerically pure L-ornidazole, i.e., with a 100% ee.

In another embodiment of this aspect, the parasitic infection is trichomonas vaginalis infection or cecal amoeba infection.

In another embodiment of this aspect, the pharmaceutical composition is a pharmaceutical dosage form containing substantially enantiomerically pure L-ornidazole.

In another embodiment of this aspect, the pharmaceutical composition is a pharmaceutical dosage selected from tablets, capsules, and injectable solutions, or the like.

In another embodiment of this aspect, the pharmaceutical dosage form is a tablet comprising substantially enantiomerically pure L-ornidazole, pregelatinized starch, sodium starch glycolate, and magnesium stearate.

In another embodiment of this aspect, the tablet dosage form comprises about 250 mg of substantially enantiomerically pure L-ornidazole, about 80 mg of pregelatinized starch, about 4 mg of sodium starch glyco late, and about 3 mg of magnesium stearate.

In another embodiment of this aspect, the tablet dosage form consists essentially of about 250 mg of substantially enantiomerically pure L-ornidazole, about 80 mg of pregelatinized starch, about 4 mg of sodium starch glycolate, and about 3 mg of magnesium stearate.

In another embodiment of this aspect, the tablet dosage form is coated by a membrane of opadry.

In another embodiment of this aspect, the pharmaceutical dosage form is a capsule comprising substantially enantiomerically pure L-ornidazole, starch, and magnesium stearate.

In another embodiment of this aspect, the capsule comprises about 250 mg of substantially enantiomerically pure L-ornidazole, about 45 mg of starch, and about 2 mg of magnesium stearate.

In another embodiment of this aspect, the capsule consists essentially of about 250 mg of substantially enantiomerically pure L-ornidazole, about 45 mg of starch, and about 2 mg of magnesium stearate.

In another embodiment of this aspect, the pharmaceutical dosage form is an injectable solution comprising substantially enantiomerically pure L-ornidazole and one or more pharmaceutically acceptable carriers selected from sodium chloride, glucose and propylene glycol.

In another embodiment of this aspect, the injectable solution comprises substantially enantiomerically pure L-ornidazole at a concentration of about 5 mg/mL and sodium chloride at a concentration of about 8.3 mg/mL.

In another embodiment of this aspect, the injectable solution consists essentially of substantially enantiomerically pure L-ornidazole at a concentration of about 5 mg/mL and sodium chloride at a concentration of about 8.3 mg/mL.

In another embodiment of this aspect, the injectable solution comprises substantially enantiomerically pure L-ornidazole at a concentration of about 5 mg/mL and glucose at a concentration of about 50 mg/mL.

In another embodiment of this aspect, the injectable solution consists essentially of substantially enantiomerically pure L-ornidazole at a concentration of about 5 mg/mL and glucose at a concentration of about 50 mg/mL.

In another embodiment of this aspect, the injectable solution comprises substantially enantiomerically pure L-ornidazole at a concentration of about 25 mg/mL and propylene glycol at a concentration of about 0.5 mg/mL.

In another embodiment of this aspect, the injectable solution consists essentially of substantially enantiomerically pure L-ornidazole at a concentration of about 25 mg/mL and propylene glycol at a concentration of about 0.5 mg/mL.

In another embodiment of this aspect, the pharmaceutical dosage form is an effervescent tablet comprising substantially enantiomerically pure L-ornidazole, sodium bicarbonate, low-substituted hydroxypropyl cellulose, sodium lauryl sulfate, microcrystalline cellulose, tartaric acid, and polyethylene glycol.

In another embodiment of this aspect, the effervescent tablet comprises about 500 mg of L-ornidazole, about 300 mg of sodium bicarbonate, about 100 mg of low-substituted hydroxypropyl cellulose, about 3.2 mg of sodium lauryl sulfate, about 440 mg of microcrystalline cellulose, about 280 mg of tartaric acid, and about 16 mg of polyethylene glycol.

In another embodiment of this aspect, the effervescent tablet consists essentially of about 500 mg of L-ornidazole, about 300 mg of sodium bicarbonate, about 100 mg of low-substituted hydroxypropyl cellulose, about 3.2 mg of sodium lauryl sulfate, about 440 mg of microcrystalline cellulose, about 280 mg of tartaric acid, and about 16 mg of polyethylene glycol.

In another embodiment of this aspect, the pharmaceutical composition is administered through an oral, an intravenous, or a vaginal delivery system.

In another embodiment of this aspect, the oral dosage of the L-ornidazole enriched pharmaceutical composition is in the range of about 10-40 mg/kg/day.

In another embodiment of this aspect, the oral dosage of the L-ornidazole enriched pharmaceutical composition is in the range of about 20-30 mg/kg/day.

In another embodiment of this aspect, the intravenous dosage of the L-ornidazole enriched pharmaceutical composition is in the range of about 5-40 mg/kg/day.

In another embodiment of this aspect, the intravenous dosage of the L-ornidazole enriched pharmaceutical composition is in the range of about 10-20 mg/kg/day.

In another embodiment of this aspect, the intravaginal dosage of the L-ornidazole enriched pharmaceutical composition is in the range of about 10-40 mg/kg/day.

In another embodiment of this aspect, the intravaginal dosage of the L-ornidazole enriched pharmaceutical composition is in the range of about 20-30 mg/kg/day.

In another embodiment of this aspect, the parasitic infection is trichomonas vaginalis infection.

In another embodiment of this aspect, the parasitic infection is trichomonas vaginalis infection, and the effervescent tablet is a vaginal effervescent tablet.

In another embodiment of this aspect, the parasitic infection is cecum amoeba infection.

In another aspect, the present invention provides a pharmaceutical composition for treatment of parasitic infections, comprising ornidazole enriched with the L-enantiomer.

In one embodiment of this aspect, the pharmaceutical composition is a pharmaceutical dosage form comprising ornidazole enriched with L-enantiomer and a pharmaceutically acceptable carrier.

In one embodiment of this aspect, the L-enantiomer enriched ornidazole in the pharmaceutical composition has at least 95.0% ee.

In another embodiment of this aspect, the L-enantiomer enriched ornidazole in the pharmaceutical composition has at least 98.0% ee.

In another embodiment of this aspect, the L-enantiomer enriched ornidazole in the pharmaceutical composition has at least 99.0% ee.

In another embodiment of this aspect, the L-enantiomer enriched ornidazole in the pharmaceutical composition has at least 99.5% ee.

In another embodiment of this aspect, the L-enantiomer enriched ornidazole in the pharmaceutical composition is substantially enantiomerically pure L-ornidazole.

In another embodiment of this aspect, the L-enantiomer enriched ornidazole in the pharmaceutical composition contains only L-ornidazole, with an undetectable level of D-ornidazole by chiral HPLC.

In another embodiment of this aspect, the L-enantiomer enriched ornidazole is substantially enantiomerically pure L-ornidazole, i.e., with a 100% ee.

In another embodiment of this aspect, the dosage form of the pharmaceutical composition is a tablet comprising ornidazole enriched with L-enantiomer, pregelatinized starch, sodium starch glycolate, and magnesium stearate.

In another embodiment of this aspect, the dosage form of the pharmaceutical composition is a tablet comprising about 250 mg of substantially enantiomerically pure L-ornidazole, about 80 mg of pregelatinized starch, about 4 mg of sodium starch glycolate, and about 3 mg of magnesium stearate.

In another embodiment of this aspect, the dosage form of the pharmaceutical composition is a tablet consisting essentially of about 250 mg of L-ornidazole, about 80 mg of pregelatinized starch, about 4 mg of sodium starch glycolate, and about 3 mg of magnesium stearate.

In another embodiment of this aspect, the tablet dosage form is coated by a membrane of opadry.

In another embodiment of this aspect, the dosage form of the pharmaceutical composition is a capsule comprising substantially enantiomerically pure L-ornidazole, starch, and magnesium stearate.

In another embodiment of this aspect, the dosage form of the pharmaceutical composition is a capsule comprising about 250 mg of substantially enantiomerically pure L-ornidazole, about 45 mg of starch, and about 2 mg of magnesium stearate.

In another embodiment of this aspect, the dosage form of the pharmaceutical composition is a capsule consisting essentially of 250 mg of L-ornidazole, about 45 mg of starch, and about 2 mg of magnesium stearate.

In another embodiment of this aspect, the dosage form of the pharmaceutical composition is an injectable solution comprising substantially enantiomerically pure L-ornidazole at a concentration of about 5 mg/mL and sodium chloride at a concentration of about 8.3 mg/mL.

In another embodiment of this aspect, the dosage form of the pharmaceutical composition is an injectable solution consisting essentially of L-ornidazole at a concentration of about 5 mg/mL and sodium chloride at a concentration of about 8.3 mg/mL.

In another embodiment of this aspect, the dosage form of the pharmaceutical composition is an injectable solution comprising substantially enantiomerically pure L-ornidazole at a concentration of about 5 mg/mL and glucose at a concentration of about 50 mg/mL.

In another embodiment of this aspect, the dosage form of the pharmaceutical composition is an injectable solution consisting essentially of L-ornidazole at a concentration of about 5 mg/mL and glucose at a concentration of about 50 mg/mL.

In another embodiment of this aspect, the dosage form of the pharmaceutical composition is an injectable solution comprising substantially enantiomerically pure L-ornidazole at a concentration of about 25 mg/mL and propylene glycol at a concentration of about 0.5 mg/mL.

In another embodiment of this aspect, the dosage form of the pharmaceutical composition is an injectable solution consisting essentially of L-ornidazole at a concentration of about 25 mg/mL and propylene glycol at a concentration of about 0.5 mg/mL.

In another embodiment of this aspect, the dosage form of the pharmaceutical composition is an effervescent tablet comprising L-ornidazole, sodium bicarbonate, low-substituted hydroxypropyl cellulose, sodium lauryl sulfate, microcrystalline cellulose, tartaric acid, and polyethylene glycol.

In another embodiment of this aspect, the dosage form of the pharmaceutical composition is an effervescent tablet comprising about 500 mg of substantially enantiomerically pure L-ornidazole, about 300 mg of sodium bicarbonate, about 100 mg of low-substituted hydroxypropyl cellulose, about 3.2 mg of sodium lauryl sulfate, about 440 mg of microcrystalline cellulose, about 280 mg of tartaric acid, and about 16 mg of polyethylene glycol.

In another embodiment of this aspect, the dosage form of the pharmaceutical composition is an effervescent tablet consisting essentially of about 500 mg of L-ornidazole, about 300 mg of sodium bicarbonate, about 100 mg of low-substituted hydroxypropyl cellulose, about 3.2 mg of sodium lauryl sulfate, about 440 mg of microcrystalline cellulose, about 280 mg of tartaric acid, and about 16 mg of polyethylene glycol.

In another embodiment of this aspect, the parasitic infection is trichomonas vaginalis infection or cecal amoeba infection.

In another embodiment of this aspect, the parasitic infection treated by the pharmaceutical composition is trichomonas vaginalis infection, and the pharmaceutical dosage form is a vaginal effervescent tablet.

In another aspect, the present invention provides methods of preparing enantiomerically pure or substantially pure L-ornidazole and D-ornidazole, as essentially disclosed and described.

In another aspect, the present invention provides methods of preparing the pharmaceutical compositions and dosage forms comprising enantiomerically pure or substantially pure L-ornidazole and D-ornidazole, as essentially disclosed and described.

The term “patient”, as used herein, refers to any mammalian animal that is inflicted with a parasitic infection(s) needing treatment, including but not limited to humans and domestic animals, for example, horses, dogs, and cats, etc.

As used herein, the singular forms “a”, “an”, and “the” include plural reference unless the context clearly dictates otherwise.

The term “about”, as used herein before a number, indicates that the number can vary by ±20%, preferably by within ±10%, and more preferably by within ±5%. When it is used before a range, it indicates that the upper and the lower limits can both vary by ±20%, preferably by within ±10%, and more preferably by within ±5%.

The phrases “L-enantiomer enriched ornidazole”, “ornidazole enriched with L-enantiomer”, or the like, refer to ornidazole products that contain L-ornidazole to D-ornidazole in at least 75:25 ratio (i.e., equal to or greater than 50% ee), preferably at least 80:20 ratio (i.e., equal to or greater than 60% ee), more preferably at least 85:15 ratio (equal to or greater than 70% ee), more preferably at least 90:10 ratio (equal to or greater than 80% ee), more preferably 95:5 ratio (equal to or greater than 90% ee), more preferably at least 97.5:2.5 ratio (equal to or greater than 95% ee), and most preferably “enantiomerically pure” or “substantially enantiomerically pure”, i.e., at least 99.0:1.0 ratio (equal to or greater than 98% ee).

The phrase “enantiomerically (or optically) pure”, “substantially enantiomerically (or optically) pure”, “substantially pure enantiomer”, or the like, as used herein, refer to either an L- or D-ornidazole product having an enantiomeric excess (ee) of at least 98.0%, preferably at least 98.5%, more preferably at least 99.0%, and most preferably at least 99.5%, based on chiral HPLC analysis, although the product could still contain an insignificant amount of other minor impurities. For example, a “substantially enantiomerically pure L-ornidazole” means an ornidazole product containing at least 99.0% of L-ornidazole and not more than 1.0% of D-ornidazole; preferably containing at least 99.25% of L-ornidazole and not more than 0.75% of D-ornidazole; more preferably 99.5% of L-ornidazole and not more than 0.5% of D-ornidazole; and most preferably 99.75% of L-ornidazole and not more than 0.25% of D-ornidazole, while the product could still contain an insignificant amount of other minor impurities. Using the method of the present invention, when an enantiomerically pure epichlorohydrin is used as starting material, both L- and D-ornidazole enantiomers can be readily prepared to have 98.0% or higher ee (i.e., equal to or greater than 99.0% optical purity).

The phrase “consisting essentially of”, as used herein, indicates that a composition does not contain other components of consequence for the intended purpose of use.

The phrase “pharmaceutically acceptable” is employed herein to refer to those compounds, materials, compositions, and/or dosage forms, which, within the scope of sound medical judgment, are suitable for use in human beings and animals commensurate with a reasonable therapeutic benefit/risk ratio.

As used herein, the phrase “pharmaceutically acceptable salts” typically refers to acid addition salts of ornidazole formed at the basic nitrogen in its imidazole ring. Examples of pharmaceutically acceptable salts of ornidazole include, but are not limited to, mineral or organic acid salts thereof. Examples of the acids that can be used to form pharmaceutically acceptable salts of L- or D-ornidazole according to the present invention include inorganic acids such as hydrochloric acid, hydrobromic acid, hydroiodic acid, sulfuric acid, and phosphoric acid, as well as organic acids, such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, carbonic acid, succinic acid, citric acid, benzoic acid, acetic acid, or related inorganic and organic acids.

The term “prodrug,” as used herein, refers to a derivative of L- or D-ornidazole that can be transformed in vivo to yield the parent compound, for example, by hydrolysis in blood. Common examples include, but are not limited to, esters of the hydroxyl group on the side chain of the L- or D-ornidazole molecule. Examples of such esters include, but are not limited to, formate, acetate, priopionate, butyrate, benzoate, or the like, as generally known to a person of skill in the art.

The term “solvate,” as used herein, means a physical association of L- or D-ornidazole with one or more, preferably one to three, solvent molecules, whether organic or inorganic. Exemplary solvates include, but are not limited to, hydrates, ethanolates, methanolates, and isopropanolates. Methods of solvation are generally known in the art.

The term “therapeutically effective amount,” as used herein, refers to the total amount of the active component that is sufficient to show a meaningful patient benefit, e.g., a sustained reduction of symptoms.

The compounds or pharmaceutical compositions of this invention can be administered for any of the uses described herein by any suitable means, for example, orally, such as tablets, capsules (each of which includes sustained release or timed release formulations), pills, powders, granules, elixirs, tinctures, suspensions, syrups, and emulsions; sublingually; bucally; parenterally, such as by subcutaneous, intravenous, intramuscular, or intrasternal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories; or intravaginally, such as intravaginal effervescent tablets. They can be administered alone, but generally will be administered with a pharmaceutical carrier selected on the basis of the chosen route of administration and standard pharmaceutical practice.

The term “pharmaceutical composition” means a composition comprising a compound of the invention in combination with at least one additional pharmaceutically acceptable carrier. A “pharmaceutically acceptable carrier” refers to media generally accepted in the art for the delivery of biologically active agents to animals, in particular, mammals, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms.

Pharmaceutically acceptable carriers are formulated according to a number of factors well within the purview of those of ordinary skill in the art. These include, without limitation, the type and nature of the active agent being formulated, the subject to which the agent-containing composition is to be administered, the intended route of administration of the composition, and the therapeutic indication being targeted. Pharmaceutically acceptable carriers include both aqueous and non-aqueous liquid media, as well as a variety of solid and semi-solid dosage forms.

The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; etc.

### Studies of Inhibitory Effects of Ornidazole Enantiomers on Central Nervous System.

In one aspect, the present invention reveals that D-ornidazole has markedly stronger inhibitory effects on the central nervous system than L-ornidazole, based on uniquely designed comparison experiments on the toxicity. The marked difference in the inhibitory effect, thus toxicity, of L- and D-ornidazoles on the CNS provides a strong benefit to using substantially pure L-ornidazole as an anti-parasitic agent.

In one experiment, the anticonvulsant effects of levo- and dextro-ornidazole against drug-induced convulsions in mice were compared. In four different animal convulsion models, mice were treated by four different seizure-inducing agents, including Remefline, Strychnine, isoniazid, thiosemicarbazide. Levo- or dextro-ornidazole was administered along with the seizure-inducing agents, and several outcomes were measured, including rate of convulsion, mortality rate, time to convulsion (latency), and time to death. Diazepam was used as a positive anticonvulsant control. Dextro-ornidazole showed clearly stronger anticonvulsant effects than levo-ornidazole in all the 4 animal models studied. For example, in the seizure model induced by Remefline, the mortality rate is 10/10 (10 out of 10 animals) for 80 mg/kg levo-ornidazole, 0/10 for 80 mg/kg dextro-ornidazole and 0/10 for the positive control diazepam. Like diazepam, stronger anticonvulsant effects of D-ornidazole indicate stronger CNS inhibition. L-Ornidazole shows remarkably lower CNS inhibition activities than D-ornidazole.

In another experiment, the effects on excitatory neurotransmitter levels by levo- and dextro-ornidazole were compared. D-ornidazole significantly decreased the excitatory neurotransmitter glutamate levels in both cerebrum and cerebellum of mice, whereas levo-ornidazole showed little effects on glutamate levels. D-ornidazole significantly decreases the level of the excitatory neurotransmitter, indicating stronger CNS inhibitory effects.

In another experiment, the effects of ornidazole enantiomers on spontaneous activities of mice were compared. The number of spontaneous activities of mice was counted by an automatic device, and the effects of ornidazole enantiomers were examined and compared. The L-ornidazole showed significantly less inhibitory effects on spontaneous activities than either D-ornidazole or the racemate.

In another experiment, the effects on motor balance and coordination of mice by ornidazole enantiomers were compared. Motor balance and coordination were tested using a modified beam walking method. Levo-ornidazole showed significantly less influence on motor balance and coordination than D- and racemate ornidazole.

In another experiment, the hypnotic effects of ornidazole enantiomers were compared.

The hypnotic effects were studied by administering levo-, dextro-ornidazole and racemate into mice with or without administering thiopental (a sleep-inducing agent). Several parameters were collected, including number of animals sleeping (loss of righting reflex), time to sleep, and time of sleeping. Levo-ornidazole showed significantly less hypnotic effects than dextro- and racemate ornidazoles in all the parameters examined, indicating lower CNS inhibitory effect.

### Subacute Toxicity Studies of Ornidazole Enantiomers.

In the studies, male and female Beagle dogs were treated at high doses of ornidazole enantiomers for 2 weeks, and then sacrificed for anatomical and pathological examinations. The toxicity of levo-ornidazole was markedly less than dextro-ornidazole and the racemate, including the toxicity symptoms, body weights and toxic effects on Purkinje cells. The D-ornidazole is so toxic that the doses had to be skipped on days 4, 8 and 11 to give animals time to recover from the toxic effects. All the doses were administered in the levo- and racemate groups. The toxicity symptoms of levo-ornidazole were reversible within 1-2 hours, whereas those caused by the D-ornidazole were not reversible. The data demonstrate that D-ornidazole is the isomer mainly responsible for the toxicity of ornidazole racemate.

Acute toxicity of L-ornidazole in mice showed that the LD₅₀ by intravenous injection was 332 mg/kg (95% CI 312˜362 mg/kg), the LD₅₀ by intraperitoneal injection of L-ornidazole was 1378 mg/kg (95% CI 1244˜1526 mg/kg), and the oral gavage LD₅₀ was 1069 mg/kg (95% CI 935.3˜1222 mg/kg). The LD₅₀ of racemic ornidazole in mice was: 306 mg/kg (95% CI 272˜346 mg/kg) by intravenous injection, 1115 mg/kg (95% CI 1026˜1212 mg/kg) by intraperitoneal injection, and 769.4 mg/kg (95% CI674.2˜878.0 mg/kg) by oral gavage. These results demonstrate that L-ornidazole has lower toxicity and better safety profile than racemic ornidazole.

### Pharmacokinetic Studies of Ornidazole Enantiomers.

In the studies, a series of pharmacokinetics studies were carried out to identify the differences between L- and D-ornidazoles. The results demonstrate that the two isomers share similar pharmacokinetics in most parameters studied. However, the data surprisingly showed that the two isomers did not inter-convert in vivo. L-ornidazole was not converted to the toxic isomer, i.e., D-ornidazole in vivo, which indicates that the benefits of L-ornidazole remained in vivo.

### Adverse Events of Ornidazole Enantiomers Observed in Clinical Studies.

The clinical trials were conducted to test if the toxicities observed in animal models would correlate with the adverse effects in humans. Double-blinded, randomized controlled human phase I and phase II clinical trials were carried out to compare L-ornidazole with racemate in both intravenous (I.V.) and oral formulations. L-ornidazole caused remarkably less adverse events (related to nervous system) than the racemate in both I.V. and oral routes. Such adverse drug events may cause non-compliance in patients being treated, which could be dangerous for life-threatening infections.

Based on the above experiments, pharmacodynamic studies on the treatment of parasitic infections in mice, including but not limited to trichomonas vaginalis infection and cecum amoeba infection, were conducted. The results showed that the efficacy of L-ornidazole in the above treatments was superior to that of D-ornidazole and racemic ornidazole.

### Dosages

The invention also provides formulations containing L-ornidazole, including oral, intravenous, and vaginal delivery systems. The oral dosage is typically about 10-40 mg/kg/day, having an optimized range of about 20-30 mg/kg/day. The intravenous dosage is typically about 5-40 mg/kg/day, having an optimized range of about 10-20 mg/kg/day. The intravaginal dosage is typically about 10-40 mg/kg/day, having an optimized range of about 20-30 mg/kg/day.

The following examples are given for the purpose of illustrating the present invention and shall not be construed as being limitations on the scope or spirit of the instant invention.

### Method of Preparation

The present invention provides a process for the preparation of ornidazole enantiomers with high optical (enantiomeric) purity. The method comprises steps of (1) reacting 2-methyl-5-nitroimidazole with enantiomerically pure or substantially pure epichlorohydrin in an organic solvent in the presence of a Lewis acid to form a condensation adduct; and (2) hydrolyzing the condensation adduct to form desired ornidazole. The product can be isolated through acidification, neutralization, and crystallization in enantiomerically pure or substantially pure form. When S-(+)-epichlorohydrin is used in the condensation reaction, S-(−)-ornidazole (i.e., L-ornidazole) is obtained; and when R-(−)-epichlorohydrin is used in the condensation reaction, R-(+)-ornidazole (i.e., D-ornidazole) is obtained. When enantiomerically pure epichlorohydrin is used as the starting material, the corresponding L- or D-ornidazole can be obtained in higher than 99.0% ee (L-:D- ratio higher than 99.5:0.5).

The preparation of (S)-(−)-ornidazole (i.e., L-ornidazole) can be represented by Scheme 1:

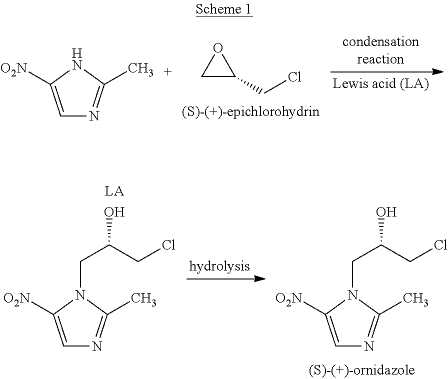

The preparation of (R)-(+)-ornidazole (D-ornidazole) can be represented by Scheme 2:

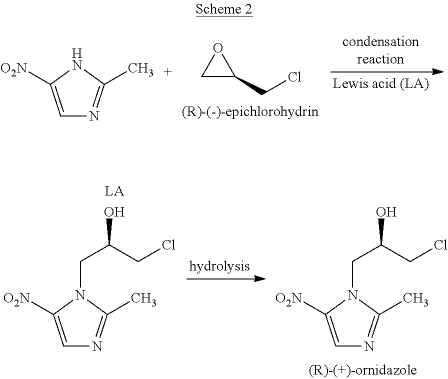

More specifically, the condensation reaction between 2-methyl-5-nitroimidazole and enantiomerically pure or substantially pure epichlorohydrin was carried out in the presence of a Lewis acid at a temperature in the range of about −10° C. to 20° C., where a preferred temperature range for the reaction is from about 0° C. to about 10° C. After complete addition of the reagents, the temperature was maintained in the range of from about 0° C. to about 20° C. for about 1 to 5 hours, where a preferred temperature range is from about 5° C. to about 10° C., and the preferred reaction time is about 2.5 hours. Then, a hydrolysis reaction was carried out at a temperature in the range from about 0° C. to about 40° C. for about 0.5 to 2 hours, where a preferred temperature range is from about 20° C. to about 30° C., and the preferred reaction time is about 1 hour. The reaction mixture was filtered and let stood for separation into layers; water and mineral acid were charged to the organic phase until obtaining a pH value between about 0.5 to about 2.0, where the preferred PH value is about 1.0, and the mixture was let stood for separation into layers. An organic solvent such as ethyl acetate and a weak base were charged to the aqueous phase until obtaining a pH value between about 6.5 to about 8.0, where the preferred pH value is between about 7.0 to about 7.5, and the mixture was let stood for separation into layers. The organic phases were combined and dried over a desiccating agent, filtered and evaporated under reduced pressure to afford a crude mass, from which the product was crystallized by adding an appropriate organic solvent to afford substantially optically pure enantiomers. When using S-(+)-epichlorohydrin as the starting material, S-(−)-ornidazole (L-ornidazole) was obtained, and when using R-(−)-epichlorohydrin as the starting material, (R)-(+)-ornidazole (D-ornidazole) was obtained. The present invention provides an improved process for the preparation of ornidazole enantiomers in a simplified process with high yield, purity, and enantiomeric excess (ee).

Suitable Lewis acids for the condensation reaction include, but are not limited to, ferric chloride, zinc chloride and aluminum chloride, or the like, where a preferred Lewis acid used for the condensation reaction is aluminum chloride.

Suitable mineral acids for the acidification process include, but are not limited to, concentrated hydrochloric acid or 40%-60% sulfuric acid by weight, wherein the preferred mineral acid used for the acidification process is concentrated hydrochloric acid or 50 wt % sulfuric acid.

Suitable weak bases for the neutralization reaction include, but are not limited to, sodium bicarbonate, triethylamine, diethylamine and ammonia water (aqueous ammonia), or the like, wherein a preferred weak base used for the neutralization reaction is ammonia water (aqueous ammonia).

Suitable organic solvents for recrystallization of ornidazole product include, but are not limited to, toluene, ethyl acetate, ethanol and methanol, or the like, wherein a preferred organic solvent for recrystallization is toluene.

According to the present invention, an optical purity of higher than 99.5% (i.e., 99.0% ee) can be readily achieved for both L- and D-enantiomers of ornidazole. Unless otherwise noted, pharmaceutical compositions or preparations used in the Examples described in this application contained >99.5% L-ornidazole and <0.5% D-ornidazole, that is, L-enantiomer in greater than 99.0% ee, or >99.5% D-ornidazole and <0.5% L-ornidazole, that is, D-enantiomer in greater than 99.0% ee.

Some illustrative, non-limiting examples of the present invention are provided below.

### EXAMPLES

### Example 1

### Preparation of L-Ornidazole

To an enamel reaction vessel (1000 L) were charged ethyl acetate (500 L) and 2-methyl-5-nitroimidazole (52.5 kg), and the mixture was cooled to 0° C. To the mixture was added aluminum chloride (80 kg) portion-wise, and the reaction mixture was maintained at a temperature below 10° C. until complete addition, and then cooled to 5° C. and maintained for 1 hour. S-(+)-Epichlorohydrin (50 L) was charged to the reaction mixture, and the temperature was maintained below 10° C. until complete addition, and then the temperature was maintained in the range of 5° C. to 10° C. for 2.5 hours. To the reaction mixture was charged ice water (300 L) gradually while maintaining temperature below 30° C. After complete addition, the temperature was maintained in the range of 20° C. to 30° C. for 1 hour, and filtered. The filtrate solution was let stand for layering. Water (200 L) and concentrated hydrochloric acid (50 L) were charged to the organic phase until obtaining a pH value at about 1.0. After separation of layers, ethyl acetate (500 L) and ammonia water were charged to the aqueous phase until obtaining a pH value between 7.0 and 7.5. After separation of layers, the organic phase was dried over MgSO₄, filtered and evaporated under pressure to afford a crude product of S-(−)-ornidazole, which was crystallized in toluene to afford (S)-(−)-ornidazole in 68% yield (29.3 kg), with [α]_{D}²⁰=−68.2° (c=1.0, CH₂Cl₂) and 99.6% chiral purity (99.2% ee).

### Example 2

### Preparation of D-Ornidazole

To an enamel reaction vessel (1000 L) were added ethyl acetate (500 Ls) and 2-methyl-5-nitroimidazole (52.5 kg), and the mixture was cooled to 0° C. Ferric chloride (80 kg) was added into the reaction vessel portion-wise, and the temperature was maintained below 10° C., and after complete addition, the reaction mixture was cooled to 5° C. and maintained for 1 hour. R-(−)-Epichlorohydrin (50 L) was charged to the reaction mixture, and the temperature was maintained below 10° C. After complete addition, the temperature was maintained in the range of 5° C. to 10° C. for 3 hours. Iced water (300 L) was added to the reaction mixture gradually while maintaining temperature below 30° C. Then, the temperature was maintained in the range of 20° C. to 30° C. for 1.5 hours. The mixture was filtered and the filtrate solution was let stand for separation of layer. Water (200 L) and concentrated hydrochloric acid (50 L) were charged to the organic phase until obtaining a pH value at bout 1.5. After separation of layers, ethyl acetate (500 L) and triethylamine were added to the aqueous phase until obtaining a pH value between 7.0 and 7.5. After separation of layers, the organic phase was dried over MgSO₄, filtered and evaporated under pressure to afford a crude product of R-(+)-ornidazole, which was crystallized in toluene to afford (R)-(+)-ornidazole in 68% yield (29.1 kg), with [α]_{D}²°=+68.4° (c=0.8, CH₂Cl₂) and 99.7% chiral purity (99.4% ee).

### Example 3

### Purification of L-Ornidazole Enantiomer

The crude product of (S)-(−)-ornidazole obtained in Example 1 (200 g, 13% impurities) and toluene (2000 mL) were added into a flask. The mixture was heated to 60° C. while stirring, and maintained for 15 minutes. The mixture was filtered while hot, and the filtrate was cooled to −5° C. for 12 hours and filtered to afford crystals (160 g, 2% impurities). The dried crystals and 75% ethanol (128 mL) were added into a flask, and the mixture was heated to 55° C. while stirring. After all the crystals dissolved, activated carbon (2 g) was added, and the mixture was stirred at 55° C. for 40 minutes. The mixture was filtered while hot, and the filtrate was cooled to 5° C. for 12 hours. The solid was filtered, washed with cold ethanol and dried to afford (S)-(−)-ornidazole (112 g, 0.2% impurities).

### Example 4

### Characterization of Ornidazole Enantiomers by Chiral HPLC

The HPLC analytical method used to quantify L-ornidazole and D-ornidazole has been qualified and validated under China GMP requirement, and the method has been used for commercial batch release in China. Agilent 1200 and a chiral column from Daicel Chiral Technologies, CHIRALCEL® OB-H 5 μm (250 mm×4.6 mm) was used with a mobile phase containing n-hexane, ethanol, acetic acid anhydride (90:10:0.1, v/v/v). The chromatography conditions are: the mobile phase flow rate of 1.0 mL/min, column temperature of 40° C., sample injection volume of 10 μL, and UV detector wavelength of 310 nm.

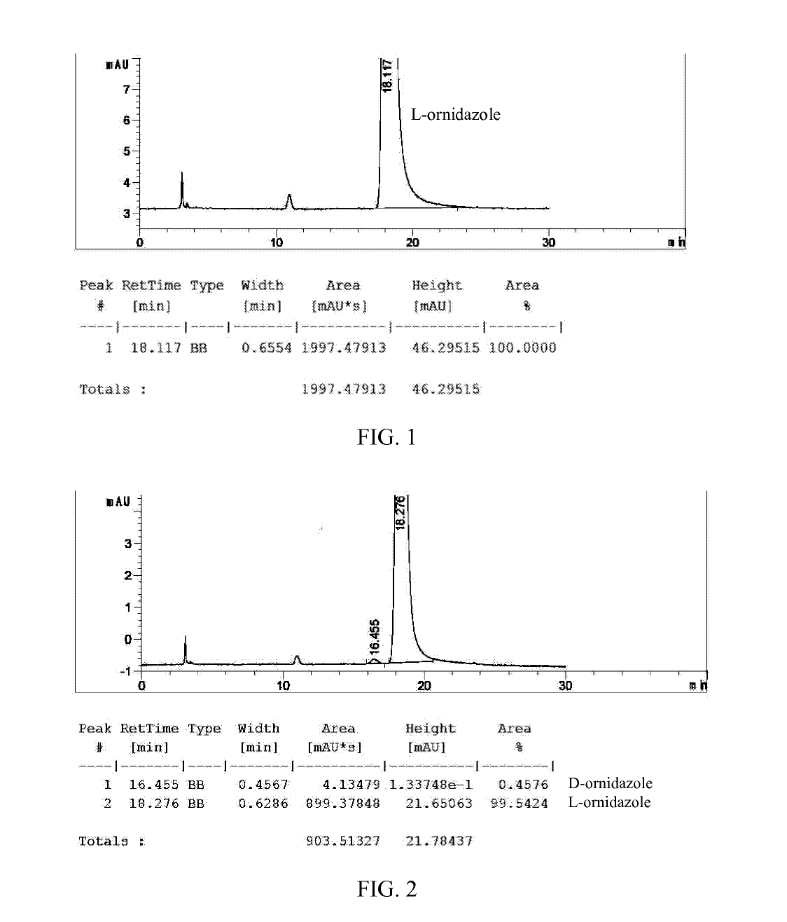

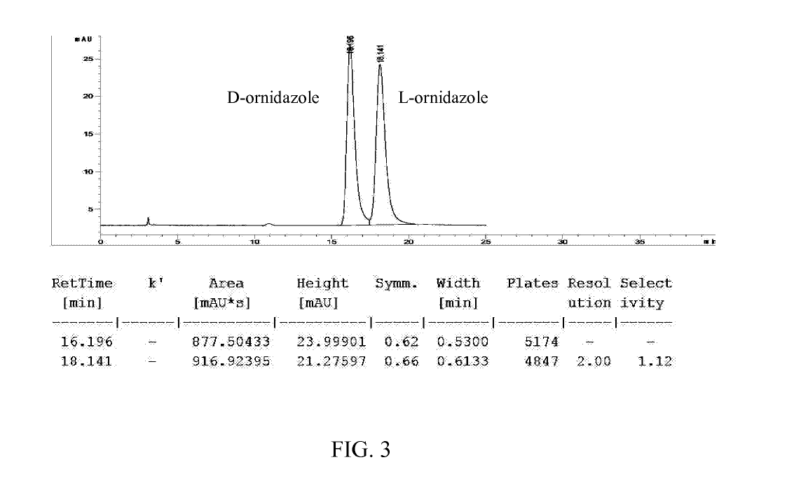

Representative examples of chromatograms of L-, D-, and racemic ornidazoles are shown in FIGS. 1-3, which include: (a) an HPLC chromatogram of an L-ornidazole sample on a chiral column (FIG. 1); (b) an HPLC chromatogram of an L-ornidazole sample on a chiral column wherein the L-ornidazole sample was spiked with 0.5% D-ornidazole (FIG. 2); and (d) an HPLC chromatogram of a racemic ornidazole sample on a chiral column (FIG. 3).

In the manufacturing quality control specifications, the chiral purity of L-ornidazole was set to be greater than 99.5%, with the impurity of D-ornidazole less than 0.5%. The above chromatograms clearly demonstrated that the pharmaceutical preparation described in this disclosure contained greater than 99.5% of L-ornidazole and less than 0.5% of D-ornidazole.

Detection of the (S)-(−)-ornidazole (L-ornidazole) and (R)-(+)-ornidazole (D-ornidazole) by HPLC.

Chromatographic conditions: (1) chromatographic column: OB-H; (2) mobile phase: isopropyl alcohol-n-hexane-methyl tertbutyl ether-glacial acetic acid (2:90:8:0.2); and (3) measurement wavelength: 314 nm. The retention time of S-(−)-ornidazole was 48.6 ml, and the retention time of R-(+)-ornidazole was 43.6 min.

### Example 5

### Characterization of Ornidazole Enantiomers by Optical Rotation

The specific rotations of S-(−)-ornidazole, R-(+)-ornidazole, and the racemate were tested on a WZZ-2s digital automatic polariscope at 20° C. and 50% of humidity.

The samples for specific rotation had been dried to constant weight over P₂O₅ at 60° C. by the Rotation photometry method described in the Pharmacopoeia of People's Republic of China 2000. The results are shown in Table 1.

TABLE 1
The results of specific rotation

| Solvent | S-(−)-ornidazole | R-(+)-ornidazole | racemates |
|---|---|---|---|
| water | −40.2° | +41.3° | 0° |
| methanol | −32.4° | +33.2° | 0° |
| ethanol | −45.3° | +45.3° | 0° |
| dichloromethane | −68.2° | +68.4° | 0° |

### Formulations of L-Ornidazole and Methods of Preparation

### Example 6

### L-Ornidazole Tablet

The following formula was used to prepare the finished dosage form of a tablet.

| Ingredient | Quantity (mg/tablet) |
|---|---|
| L-ornidazole | 250 |
| Pregelatinized Starch | 80 |
| Sodium Starch Glycolate | 4 |
| Magnesium stearate | 3 |

Procedure: The active ingredient and the excipients were sieved through a 0.15 mm sieve. L-Ornidazole and pregelatinized starch were mixed uniformly. The mixture was granulated with 8% starch slurry. Dry the granules and sieve again. Sodium starch glycolate and magnesium stearate were added to the dry granules. Compressed and film coat with 95% ethanol solution containing 8% opadry.

### Example 7

### L-Ornidazole Capsule

The following formula was used to prepare the finished dosage form of a capsule.

| Ingredient | Quantity (mg/capsule) |
|---|---|
| L-ornidazole | 250 |
| Starch | 45 |
| Magnesium stearate | 2 |

Procedure: The active ingredient and the excipients were passed through a 0.15 mm sieve. L-ornidazole and starch were mixed uniformly. The mixture was granulated with 6% starch slurry. The granules were dried and then sieved again. Magnesium stearate was added to the dry granules. The mixture was filled into capsules.

### Example 8

### L-Ornidazole Granule

The following formula was used to prepare the finished dosage form of a granule.

| Ingredient | Quantity (mg/bag) |
|---|---|
| L-ornidazole | 250 |
| Mannitol | 250 |
| Sucrose | 200 |
| Sodium Starch Glycolate | 20 |

Procedure: The active ingredient and the excipients were passed through a 0.15 mm sieve. L-ornidazole, mannitol, sucrose and sodium starch glycolate were mixed uniformly. The mixture was granulated with 8% starch slurry. Dry the granules and sieve again. Pack the granules.

### Example 9

### L-Ornidazole Infusion Formulation (NaCl)

The following formula was used to prepare the finished dosage form of an infusion.

| Ingredient | Quantity | |
|---|---|---|
| L-ornidazole | 5 | mg/ml |
| Sodium Chloride | 8.80 | mg/ml |
| Distilled water (added to) | 100 | ml |

Procedure: Dissolve L-ornidazole and sodium chloride in 40° C. distilled water. Adjust the pH of the liquid to 4.0 by 0.1 mol/L hydrochloric acid. Add 40° C. distilled water to reach the total volume. Add 0.1% active carbon to the solution. Stir well, lay aside for 15 minutes, remove active carbon by using 5 μm titanium bar. Filter through a 0.45 μm and a 0.22 μm microvoid filter film. Fill the liquid in 100 mL infusion glass bottle. Seal and sterilize by fluid steam at 100° C. for 45 minutes.

### Example 10

### L-Ornidazole Infusion Formulation (Glucose)

The following formula was used to prepare the finished dosage form of an infusion.

| Ingredient | Quantity | |
|---|---|---|
| L-ornidazole | 5 | mg/ml |
| Glucose | 50 | mg/ml |
| Distilled water (added to) | 100 | ml |

Detailed procedure: Dissolve L-ornidazole and glucose in 45° C. distilled water. Adjust the pH of the liquid to 3.5 by 0.1 mol/L hydrochloric acid. Add 45° C. distilled water to reach the total volume. Add 0.15% active carbon to the solution. Stir well; lay aside for 15 minutes; remove active carbon by 5 μm titanium bar. Filter it through a 0.45 μm and a 0.22 μm microvoid filter film. Fill the liquid in 100 ml infusion glass bottles. Seal and sterilize by fluid steam at 100° C. for 45 minutes.

### Example 11

### Injectable L-Ornidazole Formulation (Propylene Glycol)

The following formula was used to prepare the finished dosage form of an injection.

| Ingredient | Quantity | |
|---|---|---|
| L-ornidazole | 25 | mg/ml |
| Propylene glycol | 0.5 | mg/ml |
| Distilled water(added to) | 10 | ml |

Procedure: Dissolve L-ornidazole in 45° C. propylene glycol. Add some 45° C. distilled water. Adjust the pH of the liquid to 4.5 by 0.1 mol/L hydrochloric acid. Added 45° C. distilled water to reach the total volume. Add 0.1% active carbon to the solution. Stir well, lay aside for 15 minutes, remove active carbon by 5 μm titanium bar. Filter it through a 0.45 μm and a 0.22 μm microvoid filter film. Fill the liquid in 100 ml infusion glass bottles. Seal and sterilize by fluid steam at 100° C. for 45 minutes.

### Example 12

### L-Ornidazole Effervescent Tablet

The following formula was used to prepare the finished dosage form of intra-vaginal effervescent tablets.

| Ingredient | Quantity (mg/tablet) |
|---|---|
| L-ornidazole | 500 |
| Sodium bicarbonate | 300 |
| Low-substituted hydroxypropyl | 100 |
| cellulose | |
| sodium lauryl sulfate | 3.2 |
| Microcrystalline cellulose | 440 |
| Tartaric acid | 280 |
| Polyethylene Glycol | 16 |

Procedure: Pass the active ingredient and the excipients through a 0.18 mm sieve. Mix L-ornidazole, sodium lauryl sulfate, sodium bicarbonate, 75% prescription volume of microcrystalline cellulose and 80% prescription volume of low-substituted hydroxypropyl cellulose uniformly. Granulate the mixture with 60% ethanol solution, and pass through a 1.18 mm nylon sieve. Dry the moist granules at 60° C. in oven till its water content was less than 1.0%, pass the dried granules through a 1.4 mm nylon sieve. These were granules A. Measure prescribed organic acid, 25% prescription volume of microcrystalline cellulose and 20% prescription volume of low-substituted hydroxypropyl cellulose and mix them uniformly. Granulate the mixture with 60% ethanol solution, and pass them through a 1.18 min nylon sieve. The moist granules were dried at 60° C. in oven till the water was not more than 1.5%. The dried granules were passed through a 1.4 mm nylon. These were granules B. Mix granules A and B uniformly with polyethylene glycol. Compress by using a heterogeneous punch and pack.

### Comparison of Inhibitory Effects of D-ornidazole and L-ornidazole on the Central Nervous System.

### Comparison of Anticonvulsant Effects of Levo- and Dextro-Ornidazole Against Drug-Induced Convulsions in Mice.

Mice are randomized into 4 treatment groups (n=10 with half male and half female): dextro- or levo-ornidazole (dosage 40, 80, 160 mg/kg), vehicle control group (15% propylene glycol in 0.9% sodium chloride, 20 mL/kg) and the positive control group. Animals are treated with known convulsion-inducing agents, followed by the drug or vehicle control given intravenously. The dose volume is 20 mL/kg with an infusion rate of 0.2 mL/10 seconds. The anticonvulsant effects are observed and recorded (see Tables 2-6).

### Example 13

### Anticonvulsant Effects on Remefline (Dimenlini)-Induced Convulsion

TABLE 2
Comparison of anticonvulsant effects by ornidazole enantiomers
on Remefline-induced convulsion in mice (n = 10)

| Treatment | Dosage | Rate of | Mortality |
|---|---|---|---|
| Groups | (mg/kg) | Convulsion | Rate |
| Vehicle control | — | 10/10 | 10/10 |
| Levo-ornidazole | 160 | 9/10^{##} | 1/10** |
| | 80 | 10/10^{#} | 10/10^{##} |
| | 40 | 10/10 | 10/10 |
| Dextro-ornidazole | 160 | 0/10** | 0/10** |
| | 80 | 5/10* | 0/10** |
| | 40 | 10/10 | 10/10 |
| Diazepam | 2 | 0/10** | 0/10** |

^{#}P < 0.05, ^{##}P < 0.01, levo vs dextro-ornidazole;
*P < 0.05, **P < 0.01, compare with vehicle control group (chi-square test).

Administration of dextro-ornidazole to the mice having Remefline-induced convulsion resulted in significantly lower rates of convulsion and mortality than the levo-ornidazole group at the 80 and 160 mg/kg doses, indicating stronger CNS inhibitory activity of dextro-ornidazole on the mice than levo-ornidazole (Table 2). On the other hand, the high mortality rate of the mice treated with levo-ornidazole indicates the weaker, if any, CNS inhibitory activity of levo-ornidazole, i.e., lower toxicity to CNS.

### Example 14

### Anticonvulsant Effects on Strychnine-Induced Convulsion

TABLE 3
Comparison of anticonvulsant effects by ornidazole enantiomers
on Strychnine-induced convulsion in mice (Mean ± SD, n = 10)

| | Dosage | Convulsion Latency | Mortality |
|---|---|---|---|
| Treatment Groups | (mg/kg) | (min) | Rate |
| Vehicle control | — | 4.3 ± 0.5 | 10/10 |
| Levo-ornidazole | 160 | 8.6 ± 2.3** | 6/10^{#} |
| | 80 | 7.5 ± 3.3** | 10/10 |
| | 40 | 5.3 ± 2.3** | 10/10 |
| Dextro-ornidazole | 160 | 9.1 ± 2.6** | 1/10** |
| | 80 | 8.0 ± 2.0** | 7/10 |
| | 40 | 6.0 ± 0.7** | 10/10 |
| Diazepam | 2 | — | 0/10** |

**P < 0.01, compared with vehicle control.
^{#}P < 0.05, levo vs dextro-ornidazole.

Similar results were observed in the treatment of mice having Strychnine-induced convulsion. Compared with vehicle control groups, the convulsion latency correlates with the dose increase for both levo- and dextro-ornidazole groups. Compared with levo-ornidazole, dextro-ornidazole shows reduced mortality rate of the mice having Strychnine-induced convulsion (Table 3), indicating significantly more protection against Strychnine-induced convulsion. Again, these data demonstrate that dextro-ornidazole has significantly stronger CNS inhibitory effect than levo-ornidazole at the dose group of 160 mg/kg.

### Example 15

### Anticonvulsant Effects on Isoniazid-Induced Convulsion

TABLE 4
Comparison of anticonvulsant effects of ornidazole enantiomers
on isoniazid-induced convulsion in mice (Mean ± SD, n = 10)

| Treatment | Dosage | Convulsion Latency | Time to Death |
|---|---|---|---|
| Groups | (mg/kg) | (min) | (min) |
| Vehicle control | — | 37.0 ± 3.9 | 49.6 ± 6.6 |
| Levo-ornidazole | 160 | 45.0 ± 7.8* | 75.2 ± 19.0** |
| | 80 | 38.3 ± 6.3^{##} | 48.3 ± 9.6^{#} |
| | 40 | 37.1 ± 5.8^{#} | 47.6 ± 5.6 |
| Dextro-ornidazole | 160 | 48.3 ± 14.1* | 81.5 ± 15.8** |
| | 80 | 46.1 ± 2.2** | 57.0 ± 7.3* |
| | 40 | 43.2 ± 5.4* | 53.6 ± 7.2 |
| Diazepam | 2 | 57.4 ± 9.7** | 79.0 ± 12.9** |

*P(0.05) **P(0.01) compared with vehicle control.
^{#}P < 0.05, ^{##}P(0.01) levo vs dextro-ornidazole.

As compared with levo-ornidazole, dextro-ornidazole significantly prolonged the convulsion latency time and the time to death of the mice having isoniazid-induced convulsion at doses of 40 and 80 mg/kg (Table 4), indicating that dextro-ornidazole has stronger anti-convulsion effects than levo-ornidazole, indicating stronger CNS inhibitory activity.

### Example 16

### Anticonvulsant Effects on Thiosemicarbazide-Induced Convulsion

TABLE 5
Comparison of anticonvulsant effects by ornidazole enantiomers
on thiosemicarbazide-induced convulsion in mice
(Mean ± SD, n = 10)

| Treatment | Dosage | Convulsion Latency | Time to Death |
|---|---|---|---|
| Groups | (mg/kg) | (min) | (min) |
| Vehicle control | — | 67.7 ± 9.2 | 134.7 ± 25.6 |
| Levo-ornidazole | 80 | 85.0 ± 30.9^{##} | 116.7 ± 38.7^{##} |
| | 40 | 77.1 ± 16.8 | 122.6 ± 29.8 |
| Dextro-ornidazole | 80 | 121.8 ± 16.8** | 213.6 ± 56.5** |
| | 40 | 94.3 ± 25.1** | 153.9 ± 43.7 |

**P < 0.01 compared with vehicle control.
^{##}P < 0.01, levo vs dextro-ornidazole.

Similar results were observed in experiments on the mice with thiosemicarbazide-induced convulsions. As compared with levo-ornidazole, dextro-ornidazole significantly increased the convulation latency and the time to death at dose of 80 mg/kg (Table 5), indicating stronger protection from thiosemicarbazide-induced convulsion by dextro-ornidazole due to its strong inhibitory effect on the CNS.

TABLE 6
Comparison of anticonvulsant effects of ornidazole enantiomers
on thiosemicarbazide-induced convulsion in mice
(Mean ± SD, n = 10)

| Treatment | Dosage | Rate of | Mortality |
|---|---|---|---|
| Groups | (mg/kg) | Convulsion | Rate |
| Vehicle control | — | 10/10 | 10/10 |
| Levo-ornidazole | 160 | 9/10^{##} | 9/10^{##} |
| Dextro-ornidazole | 160 | 3/10** | 2/10** |
| Diazepam | 2 | 2/10** | 0/10** |

^{##}P < 0.01 levo vs dextro-ornidazole.
**P < 0.01, compared with vehicle control.

Dextro-ornidazole treatment resulted in significantly reduced rate of convulsion and death in thiosemicarbazide-induced mice (Table 6), indicating stronger protection from convulsion.

In sum, the data demonstrate marked difference of anti-convulsion activities between dextro-ornidazole and levo-ornidazole in drug-poisoned mice, including the convulsion latency, time to death, convulsion rate and mortality. Dextro-ornidazole shows stronger protection again drug-induced convulsions than levo-ornidazole, indicating the dextro-ornidazole has stronger CNS inhibitory toxicity.

### Example 17

### Comparison of the Effects of L- and D-Ornidazoles on Excitatory Neurotransmitter Levels

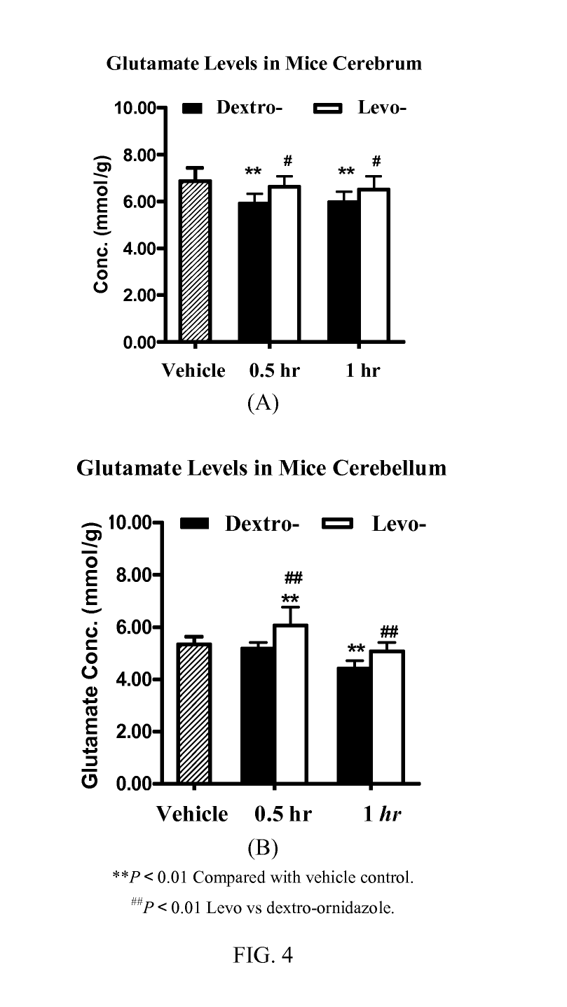

Levo-ornidazole showed little effect on the levels of glutamate in both cerebrum and cerebellum, however, dextro-ornidazole significantly decreased glutamate levels in the mice brain, which resulted in the CNS inhibitory effects (FIG. 4). The data demonstrate significant difference in effects on excitatory neurotransmitter glutamate in mice between the enantiomers

### Example 18

### Comparison of the Effects of L- and D-Ornidazoles on Spontaneous Activities of Mice

Mice weighing 18-22 g, half male and half female, were placed in the laboratory 24 hours before the test to adapt to the environment. The room temperature was controlled at 24±2° C. Animals were fasted for 8 hours before testing, and then placed in a transparent cage. The number of spontaneous activities of mice was counted by an automatic device (model XZ-4 by Drug Research Institute of China Academy of Medical Sciences). Mice were pre-screened by counting their spontaneous activities for five minutes and unqualified mice were excluded. A total of 110 mice were selected and divided into 11 groups with 10 mice in each group. There was no significant difference in average number of spontaneous activities among the 11 groups before drug administration. After drugs were given intravenously, spontaneous activities were counted at five time points after each dose was given.

TABLE 7
Effects of L- and D-ornidazoles on spontaneous activities of mice (Mean ± SD, n = 10)

| | | Spontaneous Activities (per 5 min) | | | | | |
|---|---|---|---|---|---|---|---|
| | Dose | | Post-Dose | | | | |
| Groups | (mg/kg) | Pre-Dose | 0.5 h | 1 h | 2 h | 3 h | 4 h |
| Vehicle | — | 186 ± 27 | 103 ± 36 | 106 ± 40 | 66 ± 26 | 72 ± 43 | 64 ± 38 |
| Levo (L)- | | 187 ± 34 | 89 ± 48 | 75 ± 40 | 69 ± 29 | 63 ± 16 | 87 ± 43 |
| Dextro (D)- | 40 | 190 ± 29 | 52 ± 23# | 44 ± 19 | 35 ± 20## | 47 ± 33 | 62 ± 31 |
| Racemate (R)- | | 189 ± 26 | 79 ± 26 | 56 ± 37 | 63 ± 23 | 62 ± 24 | 72 ± 35 |
| p value | D- vs L- | 0.7890 | 0.0370 | 0.1273 | 0.0046 | 0.0875 | 0.2003 |
| | R- vs L- | 0.828 | 0.788 | 0.313 | 0.649 | 0.757 | 0.422 |
| Levo- | 80 | 188 ± 24 | 59 ± 27 | 47 ± 30 | 44 ± 25 | 58 ± 36 | 64 ± 36 |
| Dextro- | | 187 ± 30 | 25 ± 15# | 16 ± 21## | 17 ± 15## | 27 ± 17# | 34 ± 17# |
| Racemate | | 188 ± 32 | 48 ± 35 | 26 ± 19 | 32 ± 21 | 55 ± 27 | 61 ± 20 |
| p value | D- vs L- | 0.8926 | 0.0150 | 0.0035 | 0.0050 | 0.0287 | 0.0463 |
| | R- vs L- | 0.888 | 0.403 | 0.106 | 0.237 | 0.959 | 0.795 |
| Levo- | 160 | 189 ± 26 | 40 ± 31 | 45 ± 25 | 38 ± 18 | 53 ± 23 | 58 ± 26 |
| Dextro- | | 186 ± 19 | 0 ± 0## | 0 ± 0## | 0 ± 0## | 3 ± 3## | 5 ± 5## |
| Racemate | | 189 ± 19 | 29 ± 17 | 22 ± 14# | 21 ± 12# | 30 ± 13# | 51 ± 23 |
| p value | D- vs L- | 0.8529 | 0.0000 | 0.0000 | 0.0000 | 0.0000 | 0.0000 |
| | R- vs L- | 0.9426 | 0.4569 | 0.0100 | 0.0222 | 0.0160 | 0.6484 |
| Chlorpromazine | 3 | 190 ± 19 | 5 ± 2 | 6 ± 4 | 13 ± 6 | 21 ± 9 | 35 ± 9 |

#P < 0.05, ##P < 0.01, compared with levo-ornidazole.
Differences among groups using analysis of variance, differences between the two groups using Dunnett test.

Levo-, dextro-ornidazole and racemate were studied and compared in nine treatment groups at three different dose levels of 40, 80 and 160 mg/kg. Chlorpromazine (3 mg/kg) was used as the positive control. The dose volume was 0.2 mL/10 gram and infusion rate was 0.2 mL/10 seconds.

The effects of the doses of L- and D-ornidazoles on inhibition of the spontaneous activities of mice were observed. Levo-ornidazole showed significantly less inhibitory effects than both dextro-ornidazole (at the doses of 40, 80 and 160 mg/kg) and the racemate (at the dose of 160 mg/kg) (Table 7).

### Example 19

### Comparison of the Effects of L- and D-ornidazoles on Motor Balance and Coordination

TABLE 8
Effects on motor balance and coordination by ornidazole enantiomers (n = 10)

| | | Number of Animals Fall Off | | | | | |
|---|---|---|---|---|---|---|---|
| Treatment | Dosage | | Time post-dose (hours) | | | | |
| Groups | (mg/kg) | Pre-dose | 0.5 | 1 | 2 | 3 | 4 |
| Vehicle | — | 0 | 0 | 0 | 0 | 0 | 0 |
| Control | | | | | | | |
| Levo(L)- | 40 | 0 | 0 | 1 | 0 | 0 | 0 |
| Dextro(D)- | | 0 | 0 | 1 | 2 | 3 | 0 |
| Racemate(R)- | | 0 | 1 | 0 | 0 | 0 | 0 |
| p-Value | D- vs L- | 1.000 | 1.000 | 0.3048 | 0.4747 | 0.2126 | 1.000 |
| p-Value | R- vs L- | 1.000 | 1.000 | 1.000 | 1.000 | 1.000 | 1.000 |
| Levo(L)- | 80 | 0 | 0 | 0 | 0 | 1 | 1 |
| Dextro(D)- | | 0 | 3 | 3 | 2 | 1 | 0 |
| Racemate(R)- | | 0 | 0 | 0 | 2 | 0 | 0 |
| p-Value | D- vs L- | 1.000 | 0.2126 | 0.2126 | 0.4747 | 0.3048 | 1.000 |
| p-Value | R- vs L- | 1.000 | 1.000 | 1.000 | 0.4747 | 1.000 | 1.000 |
| Levo(L)- | 160 | 0 | 0 | 0 | 3 | 2 | 1 |
| Dextro(D)- | | 0 | 10## | 10## | 10## | 10## | 9## |
| Racemate(R)- | | 0 | 10## | 8## | 6 | 4 | 3 |
| p-Value | D- vs L- | 1.000 | 0.00006 | 0.00006 | 0.0040 | 0.0012 | 0.0005 |
| p-Value | R- vs L- | 1.000 | 0.00006 | 0.00116 | 0.3687 | 0.6256 | 0.5762 |
| Chlorpromazine | 3 | 0 | 10 | 10 | 10 | 9 | 5 |

##P < 0.01, compared with levo-ornidazole (simplified direct probability method).

Motor balance and coordination were tested using a modified beam walking method. Mice were put on a smooth beam tilted at an angle of 70 degree. Mice passed the test by standing on the beam for 0.5 minute without falling off. All the mice were pre-screened before the studies, and a total of 110 mice were selected by passing the beam test in 3 consecutive tries. The selected mice were divided into 11 groups with 10 mice in each group (half male and half female). At certain time points after drug administration, mice were tested on the beam for 3 consecutive times. A mouse was counted as failing to the test if it fell off the beam for 2 or more times.

Levo-ornidazole, dextro-ornidazole and racemate were studied and compared in nine treatment groups at three different dose levels of 40, 80 and 160 mg/kg. The dose volume was 0.2 mL/10 gram and infusion rate was 0.2 mL/10 seconds. Chlorpromazine was used as the positive control.

As shown in Table 8, levo-ornidazole showed significantly less influence on motor balance and coordination than dextro- and racemate ornidazole at the dose of 160 mg/kg.

### Example 20

### Comparison of the Hypnotic Effects of Ornidazole Enantiomers

A total of 110 mice were divided into 11 groups with 10 mice in each group (half male and half female) and animals were fasted for 8 hours before study. Thirty minutes after drug administration, the number of animals losing righting reflex (LORR) was recorded. Levo-, dextro-ornidazole and racemate were studied and compared in nine treatment groups at three different dose levels of 40, 80 and 160 mg/kg. Sodium pentobarbital was used at 3 mg/kg as the positive control. The dose volume was 0.2 mL/10 gram, and the infusion rate was 0.2 mL/10 seconds.

At the dose of 160 mg/kg, none of the mice lost righting reflex in the levo-ornidazole group, however, a total of 10 and 6 out of 10 mice lost righting reflex respectively in the dextro-ornidazole and the racemate groups, indicating stronger hypnotic effects by dextro- and racemate ornidazole than L-ornidazole (Table 9).

TABLE 9
Hypnotic effect of ornidazole enantiomers

| | | | Number of | |
|---|---|---|---|---|
| | | | animals | |
| | | | Loss of | p value |
| | | | Righting | (comparing |
| Treatment | Dosage | Animal | Flex 30 min | with Levo- |
| Groups | (mg/kg) | Number | Post-dose | ornidazole) |
| Vehicle Control | — | 10 | 0 | |
| Levo- | 40 | 10 | 0 | |
| | 80 | 10 | 0 | |
| | 160 | 10 | 0 | |
| Dextro- | 40 | 10 | 0 | 1.000 |
| | 80 | 10 | 0 | 1.000 |
| | 160 | 10 | 10**^{##} | 0.00006 |
| Racemate | 40 | 10 | 0 | 1.000 |
| | 80 | 10 | 0 | 1.000 |
| | 160 | 10 | 6*^{#} | 0.01239 |
| Pentobarbital | 40 | 10 | 10** | |

*P < 0.05, **P < 0.01, treatment groups vs vehicle controls (simplified direct probability method).
^{##}P < 0.01, compared with the same dosage group of levo-ornidazole (simplified direct probability method). Propylene glycol in 0.9% sodium chloride was used by a vehicle control.

### Example 21

### Effects of Ornidazole Enantiomers on Thiopental-Induced Hypnosis

A total of 70 mice were divided into 7 groups with 10 mice in each group (half male and half female), and animals were fasted for 8 hours before study. Vehicle control and ornidazole enantiomers were injected intraperitoneal (IP), after 30 minutes, sodium thiopental was then given IP: at 40 mg/kg. The time to lose and regain righting reflex was recorded (Table 10).

Levo-, dextro-ornidazole and racemate are studied and compared in 6 treatment groups at 2 different dose levels of 40 and 80 mg/kg. The dose volume is 0.2 mL/10 gram and infusion rate is 0.2 mL/10 seconds.

TABLE 10
Effects of ornidazole enantiomers on thiopental-induced
hypnosis (Mean ± SD, n = 10)

| | | | Time to Lose | |
|---|---|---|---|---|
| | | Number | Righting | |
| | Dosage | of | Reflex | Sleeping Time |
| Treatment Groups | (mg/kg) | Animals | (min) | (min) |
| Vehicle Control | — | 10 | 2.6 ± 0.5 | 5.9 ± 2.9 |
| Levo (L)- | | 10 | 2.4 ± 0.2 | 9.7 ± 6.9 |
| Dextro (D)- | 40 | 10 | 2.1 ± 0.4^{#} | 120.0 ± 93.7^{##} |
| Racemate (R)- | | 10 | 2.1 ± 0.3^{#} | 74.0 ± 51.3^{##} |
| p-Value | D- vs L- | | 0.04182 | 0.00030 |
| p-Value | R- vs L- | | 0.02158 | 0.00051 |
| Levo (L)- | | 10 | 2.3 ± 0.5 | 73.1 ± 47.9 |
| Dextro (D)- | 80 | 10 | 1.8 ± 0.3## | 275.9 ± 54.3## |
| Racemate (R)- | | 10 | 1.9 ± 0.4# | 106.6 ± 99.2# |
| p-Value | D- vs L- | | 0.00804 | 0.00038 |
| p-Value | R- vs L- | | 0.04259 | 0.03266 |

#P < 0.05, ##P < 0.01, compared with levo-ornidazole.
Differences among groups using analysis of variance, differences between the two groups using Dunnett test. Propylene glycol in 0.9% sodium chloride was used by a vehicle control.

Dextro-ornidazole and racemate showed significantly stronger synergistic effect than levo-ornidazole at both dose levels tested on thiopental-induced hypnosis in mice.

### Example 22

### Subacute Toxicity Study

Male and female Beagle dogs were kept in laboratory conditions for 1 week to observe general health conditions. Animals with similar weights and in good health were selected and then divided randomly into 4 treatment groups: levo-, dextro-ornidazole, racemate and vehicle control group. Animals were treated at dose of I.V. 200 mg/kg for ornidazole enantiomers, or 10 mL/kg for vehicle control for 2 weeks, then sacrificed for anatomical and pathological examinations.

### Example 23

### Comparison of Symptoms of Toxicity of Ornidazole Enantiomers in Beagle Dogs

The symptoms of toxicity of levo-ornidazole are markedly less than dextro-ornidazole and the racemate (Table 11). The toxicity symptoms of levo-ornidazole are reversible within 1-2 hours and not reversible for dextro-ornidazole. It clearly demonstrates that dextro-ornidazole is the isomer mainly responsible for the toxicity.

TABLE 11
Comparison of symptoms of toxicity of ornidazole enantiomers
in Beagle dogs.

| Treatment | Toxicity | | |
|---|---|---|---|
| Groups | Symptoms | Severity | Recovery |
| Levo- | Salivating, vomiting, | Mild | Symptoms disappear |
| | fecal and urine | | 1-2 hours after |
| | incontinence | | administraion |
| Dextro- | Vomiting, salivating, | Severe | Because of the severe |
| | Limb weakness, | | toxicity symptoms, |
| | unable to stand, | | doses of dextro- |
| | convulsions | | ornidazole are skipped |
| | | | on days 4, 8 and 11. |
| | | | Doses are not skipped |
| | | | on the other 2 groups. |
| | | | On day 14, all animals |
| | | | are unable to stand with |
| | | | Intermittent convulsions. |
| Racemate | Vomiting, salivating, | Moderate | Symptoms worsen with |
| | Limb weakness, | | more doses given and |
| | difficulty in standing | | the recovery time |
| | | | prolongs. |

### Example 24

### Effects of Levo-, Dextro- and Racemic Ornidazoles on Body Weights of Beagle Dogs by I.V. Administration

The effects of levo-, dextro- and racemic Ornidazoles on body weight of Beagle dogs by I.V. administration were studied, using propylene glycol in 0.9% sodium chloride as a vehicle control. The body weight of Beagle dogs treated with L-ornidazole decreased significantly slower than those of Beagle dogs treated with either D-ornidazole or a racemic mixture, while the D-ornidazole treated Beagle dogs lost body weight the fastest (FIG. 5). It is noteworthy that D-ornidazole was so toxic that its doses had to be skipped on days 4, 8 and 11 in order to give animals time to recover from the toxic effects. All the doses were given to the levo- and racemate groups.

### Example 25

### Effects of Ornidazole Enantiomers on Purkinje Cells in Cerebellar Slices in Beagle Dogs

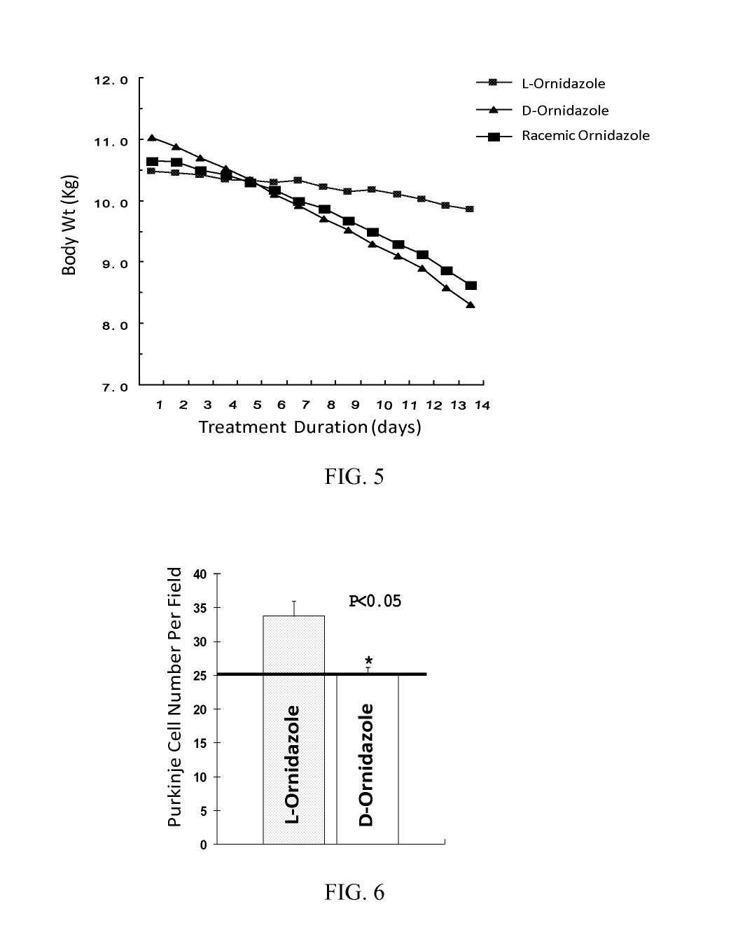

The effects of L- and D-ornidazoles on the number of Purkinje cells in cerebellar slices of Beagle dogs (mean±SD, n=4) were studied at the dose level of 200 mg/Kg, using propylene glycol in 0.9% sodium chloride as a vehicle control. Quantitative data were analyzed by group test and qualitative data by simplified direct probability method. A 10×10 area under low magnification was used to count cerebellar Purkinje cells. The results showed that treatment by dextro-ornidazole resulted in significantly more loss of the cerebellar Purkinje cells as compared with treatment by levo-ornidazole (FIG. 6).

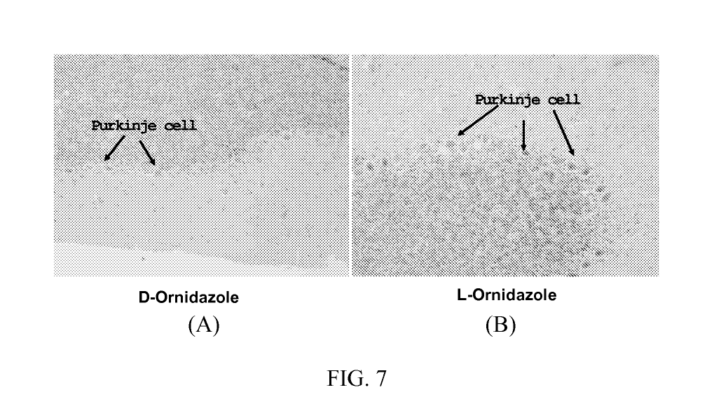

After 14 days of treatment, dextro-ornidazole resulted in not only decreased number of Purkinje cells, but also Purkinje cell denaturing and morphology change, whereas levo-ornidazole did not cause significant change on Purkinje cells (FIG. 7).

In conclusion, the subacute toxicity study demonstrates that levo-ornidazole is significantly less toxic than dextro-ornidazole and the racemate. The dextro-ornidazole is the main cause for toxicity on nervous system.

### Example 26

### Pharmacokinetics Studies

The properties of stereoisomers in vivo are generally considered to be complex and unpredictable, and many aspects need to be considered and studied including the processes of absorption, distribution, metabolism and elimination. A series of pharmacokinetics studies have been carried out to characterize the following in vivo pharmacokinetics-related behaviors of the two enantiomers and the findings are summarized in below:

a) Similar pharmacokinetics for both levo-ornidazole and dextro-ornidazole in rats.
b) Similar elimination patterns for both levo-ornidazole and dextro-ornidazole in urine in rats.
c) Similar metabolites in rat urine.
d) Similar plasma protein binding for both levo-ornidazole and dextro-ornidazole.
e) Similar distribution of both levo-ornidazole and dextro-ornidazole in different region of rat brain. Similar permeability and uptake through blood brain barrier.
f) Similar metabolism in vivo for both levo-ornidazole and dextro-ornidazole.

Despite all the tests showed similar pharmacokinetic behaviors for these L- and D-ornidazoles, to the present inventors' surprise, the data demonstrate that the L- and D-enantiomers do not inter-convert in vivo.

Chiral inversion (or “inter-conversion”) is known as that one enantiomer of a drug is converted into its antipode. Wsól, V., et al. suggested that chiral inversion is much more frequent than it can be realized (Wsól, V., et al., Curr. Drug Metab. 5(6):517-33 (2004)). Studies were conducted to test if the L- and D-enantiomers of ornidazole would inter-convert in vivo.

The two enantiomers and the racemate ornidazole were given separately to Beagle dogs (n=4) intravenously at a dosage of 15 mg/kg. Blood samples were collected at different time points up to 16 hours after the drug infusion. Plasma samples were mixed with methanol/isopropanol (1:1, v/v), vortexed, spun down, and then analyzed by HPLC. Plasma drug concentrations were generated (see Tables 12-14).

TABLE 12
Plasma drug concentrations (μg/mL) after administration of L-ornidazole

Time (hr)

| No | | 0.083 | 1 | 2 | 4 | 6 | 8 | 12 | 16 |
|---|---|---|---|---|---|---|---|---|---|
| 1 | L- | 13.24 | 9.47 | 7.78 | 7.51 | 5.05 | 3.99 | 3.24 | 1.95 |
| | D- | — | — | — | — | — | — | — | — |
| 2 | L- | 12.17 | 11.54 | 8.79 | 6.46 | 4.70 | 3.15 | 2.39 | 1.40 |
| | D- | — | — | — | — | — | — | — | — |
| 3 | L- | 10.37 | 9.94 | 8.27 | 5.62 | 4.48 | 3.26 | 2.11 | 1.19 |
| | D- | — | — | — | — | — | — | — | — |
| 4 | L- | 13.95 | 9.45 | 7.62 | 5.73 | 3.99 | 2.99 | 1.91 | 1.06 |
| | D- | — | — | — | — | — | — | — | — |

— below detection limit of 0.25 μg/mL.

TABLE 13
Plasma drug concentrations (μg/mL) after
administration of I.V. D-ornidazole

| | | Time (hr) | | | | | | | |
|---|---|---|---|---|---|---|---|---|---|
| No | | 0.083 | 1 | 2 | 4 | 6 | 8 | 12 | 16 |
| 1 | L- | — | — | — | — | — | — | — | — |
| | D- | 15.03 | 11.12 | 9.47 | 6.57 | 4.09 | 2.63 | 1.34 | 0.98 |
| 2 | L- | — | — | — | — | — | — | — | — |
| | D- | 12.21 | 11.44 | 8.31 | 5.20 | 3.50 | 2.32 | 1.01 | 0.44 |
| 3 | L- | — | — | — | — | — | — | — | — |
| | D- | 14.24 | 10.20 | 8.52 | 4.90 | 3.32 | 2.18 | 0.87 | 0.42 |
| 4 | L- | — | — | — | — | — | — | — | — |
| | D- | 15.15 | 7.52 | 7.44 | 4.98 | 2.72 | 1.61 | 0.55 | — |

— below detection limit of 0.25 μg/mL.

TABLE 14
Plasma drug concentrations (μg/mL) after
administration of racemate ornidazole

| | | Time (hr) | | | | | | | |
|---|---|---|---|---|---|---|---|---|---|
| No | | 0.083 | 1 | 2 | 4 | 6 | 8 | 12 | 16 |
| 1 | L- | 6.54 | 6.17 | 4.46 | 3.55 | 2.60 | 2.13 | 1.71 | 1.32 |
| | D- | 7.47 | 6.44 | 4.68 | 3.06 | 2.13 | 1.46 | 0.97 | 0.51 |
| 2 | L- | 7.40 | 5.41 | 4.53 | 3.47 | 3.19 | 2.63 | 1.41 | 0.88 |
| | D- | 8.44 | 5.91 | 4.49 | 2.74 | 1.98 | 1.34 | 0.50 | — |
| 3 | L- | 6.22 | 4.92 | 4.17 | 2.68 | 2.12 | 1.49 | 1.24 | 0.50 |
| | D- | 7.03 | 5.49 | 4.23 | 2.42 | 1.60 | 1.00 | 0.52 | — |
| 4 | L- | 8.23 | 5.78 | 4.56 | 2.98 | 2.28 | 2.22 | 1.42 | 0.37 |
| | D- | 9.22 | 6.13 | 4.58 | 2.62 | 1.53 | 1.82 | 0.24 | — |

— below detection limit of 0.25 μg/mL.

The results in Tables 12-14 showed that D-ornidazole was not detected in vivo when L-ornidazole was administered intravenously; neither was L-ornidazole when D-ornidazole was administered. Therefore, chiral inversion did not occur when a single enantiomer was administered.

### Example 27

### Pharmacodynamic Study of Trichomonas Vaginalis Infection

Male ICR mice were injected intraperitoneally with liquid containing 0.4 ml 3×10⁶ trichomonas vaginalis (trichomonas vaginalis from clinical patients). The mice were randomly divided into 19 groups, each with 10. The mice in solvent control group were given blank solutions by tail vein injection. The treatment groups were injected intravenously at 2, 24, 48, and 72 hours after infection with active drugs. The animals were killed five days after infection. Washing offal, fluid was centrifuged, and microscopically examined for life larvae. Autopsy was performed to determine whether there was any visceral and abdominal abscess, microscopic examination was conducted to determine the number of live trichomoniasis in the abscess. Results showed multiple offal and abdominal small abscess formation in the solvent control group mice. The abscess formation was inhibited in the treatment groups□ and the number of live trichomoniasis was reduced. Calculate the dosage of 50% (ED₅₀) and 90% (ED₉₀) inhibition rate. The results were as shown in Table 15.

TABLE 15
Pharmacodynamic data on treatment of Trichomonas
vaginalis infection

| Drug | ED₅₀ | 95% CI | ED₉₀ | 95% CI |
|---|---|---|---|---|
| L-ornidazole | 9.1 | 4.8~17.4 | 32.6 | 17.0~61.7 |
| D-ornidazole | 14.1 | 8.3~24.0 | 54.5 | 32.4~93.3 |
| Racemic | 11.3 | 5.5~22.9 | 43.8 | 21.4~89.1 |
| ornidazole | | | | |

The lower ED₅₀ and ED₉₀ values of L-ornidazole showed that it is superior to D-ornidazole and racemic ornidazole in the treatment of trichomonas vaginalis.

### Example 28

### Pharmacodynamic Study in the Treatment of Cecum Amoeba Infection

Male ICR mice were injected with liquid containing about 200,000 units of amoeba dysentery in the cecum. The mice were randomly divided into 19 groups, each with 10. The mice in solvent control group were given blank solutions by tail vein injection. The treatment groups were injected intravenously at 24, 48, 72 hours after infection with active drugs. The animals were killed six days after infection. Intestinal mucosal biopsies were done, and comparative studies of different stages of the amoeba were studied under microscope. Results showed the growth of Amebic dysentery on the cecum in the solvent control group, and the inhibition and killing of the Amebic dysentery in the treatment groups. Calculated the dosage of 50% (ED₅₀) and 90% (ED₉₀) inhibition rate. The results were as shown in Table 16.

TABLE 16
Pharmacodynamic data on the treatment of
cecum amoeba infection

| Drug | ED₅₀ | 95% CI | ED₉₀ | 95% CI |
|---|---|---|---|---|
| L-ornidazole | 10.5 | 5.4~20.4 | 41.2 | 21.4~81.3 |
| D-ornidazole | 13.3 | 8.3~20.8 | 64.4 | 40.7~102.3 |
| Racemic ornidazole | 11.4 | 5.6~22.9 | 55.2 | 27.5~109.6 |

The lower ED₅₀ and ED₉₀ data showed that L-ornidazole was superior to D-ornidazole and racemic ornidazole in the treatment of cecum amoeba infection.

These experiments show that L-ornidazole has better efficacy, pharmacokinetics characteristics, and lower central nervous system toxicity in the treatment of parasitic infections (including trichomonas vaginalis infection and cecum amoeba infection in mice) than D-ornidazole and racemic ornidazole. The L-isomer will be the more desired formulation in the clinical practice.

### Adverse Events of L- and D-Ornidazole Observed in Clinical Studies

### Example 29

### Phase I Clinical Trials of Intravenous (I.V.) Racemate and Levo-Ornidazole

The data from the phase I clinical trials demonstrate that racemate ornidazole causes markedly more adverse events than levo-ornidazole at a regular human dose level of 1 gram (Table 17).

TABLE 17
Adverse events observed for intravenous racemate and
levo-ornidazole in human phase I clinical trials

# of Patients
Reporting
Adverse Events/
Total Patients

| Dosage | Levo- | Racemate |
|---|---|---|
| 0.5 g, I.V., qd × 1d | 0/9 | / |
| 1.0 g, I.V., qd × 1d | 0/10 | 5/10 |
| 1.25 g, I.V., qd × 1d | 0/10 | / |
| 1.5 g, I.V., qd × 1d | 2/10 | / |

### Example 30

### Adverse Events Observed during Phase II Clinical Trials of Intravenous (I.V.) Levo-Ornidazole and Racemate

A double-blinded randomized controlled trial was carried out to compare the adverse events between the I.V. levo-ornidazole and racemate treatment groups. The total incidence rate of adverse events was 1.47% (2 patients out of 136 patients, with 3 adverse events reported) for levo-ornidazole, and 21.58% (30 patients out of 139 patients, with 42 adverse events reported) for racemate ornidazole with a p-value <0.01. The high rate of adverse events observed from the treatment by the racemic drug caused patient treatment non-compliant, which could be dangerous for life-threatening infections. However, levo-ornidazole caused remarkably less adverse events than the racemate, as shown in Table 18.

TABLE 18
Adverse events (AE) observed for intravenous Levo- and racemate ornidazole in
phase II human clinical trials.

| Adverse Event | Treatment | # of Patients | # of Patients | Total | Incidence | |
|---|---|---|---|---|---|---|
| (AE) | Groups | with AE | w/o AE | Patients | (%) | p-Value |
| Vertigo | Racemate | 17 | 122 | 139 | 12.23^{##} | 0.0000 |
| | Levo- | 0 | 136 | 136 | 0.00 | |
| Dizziness | Racemate | 5 | 134 | 139 | 3.60 | 0.0603 |
| | Levo- | 0 | 136 | 136 | 0.00 | |
| Headache | Racemate | 1 | 138 | 139 | 0.72 | 1.0000 |
| | Levo- | 0 | 136 | 136 | 0.00 | |
| Hypersomnia | Racemate | 10 | 129 | 139 | 7.19^{##} | 0.0017 |
| | Levo- | 0 | 136 | 136 | 0.00 | |
| Hypodynamia | Racemate | 1 | 138 | 139 | 0.72 | 1.0000 |
| | Levo- | 0 | 136 | 136 | 0.00 | |
| Vomiting | Racemate | 1 | 138 | 139 | 0.72 | 1.0000 |
| | Levo- | 0 | 136 | 136 | 0.00 | |
| Nausea | Racemate | 1 | 138 | 139 | 0.72 | 1.0000 |
| | Levo- | 0 | 136 | 136 | 0.00 | |
| Dry mouth | Racemate | 2 | 137 | 139 | 1.44 | 0.4982 |
| | Levo- | 0 | 136 | 136 | 0.00 | |
| Appetite drop | Racemate | 0 | 139 | 139 | 0.00 | 0.4945 |
| | Levo- | 1 | 135 | 136 | 0.74 | |
| Stomach | Racemate | 1 | 138 | 139 | 0.72 | 1.0000 |
| discomfort | Levo- | 0 | 136 | 136 | 0.00 | |
| WBC decline | Racemate | 3 | 136 | 139 | 2.16 | 1.0000 |
| | Levo- | 2 | 134 | 136 | 1.47 | |

^{#}P < 0.05, ^{##}P < 0.01, levo-ornidazole comparing with racemate.
AE: Adverse event

### Example 31

### Adverse Events of Oral Levo-Ornidazole and Racemate Tablets in Phase II Clinical Trials Conducted by Departments of Stomatology and Obstetrics/Gynecology

Double-blinded randomized controlled trials were carried out to compare the adverse events of oral levo-ornidazole with racemate treatment groups. Two separate studies were conducted for patients with infections from either department of stomatology or obstetrics/gynecology.

Data from Departments of Stomatology

The total incidence rate of adverse events among patients from department of stomatology was 3.60% (5 out of 139 patients) for levo-ornidazole, and 17.73% (25 out of 141 patients) for racemate ornidazole with a p-value <0.01 (Table 19).

TABLE 19
Adverse events observed for oral levo- and racemate ornidazole in phase II human
clinical trials conducted among patients from departments of stomatology.

| Adverse Event | Treatment | # of Patients | # of Patients | Total | Incidence | |
|---|---|---|---|---|---|---|
| (AE) | Groups | with AE | w/o AE | Patients | (%) | p-Value |
| Hypersomnia | Levo- | 0 | 139 | 139 | 0.00^{#} | 0.0296 |
| | Racemate | 6 | 135 | 141 | 4.26 | |
| Vertigo | Levo- | 0 | 139 | 139 | 0.00 | 0.4982 |
| | Racemate | 2 | 139 | 141 | 1.42 | |
| Palpitation | Levo- | 0 | 139 | 139 | 0.00 | 1.0000 |
| | Racemate | 1 | 140 | 141 | 0.71 | |
| Hypodynamia | Levo- | 0 | 139 | 139 | 0.00 | 1.0000 |
| | Racemate | 1 | 140 | 141 | 0.71 | |
| Bloating | Levo- | 0 | 139 | 139 | 0.00 | 1.0000 |
| | Racemate | 1 | 140 | 141 | 0.71 | |
| Vomiting | Levo- | 0 | 139 | 139 | 0.00 | 1.0000 |
| | Racemate | 1 | 140 | 141 | 0.71 | |
| Sourregurgitation | Levo- | 0 | 139 | 139 | 0.00 | 1.0000 |
| | Racemate | 1 | 140 | 141 | 0.71 | |
| Nausea | Levo- | 4 | 135 | 139 | 2.88 | 1.0000 |
| | Racemate | 4 | 137 | 141 | 2.84 | |
| Drowsy | Levo- | 0 | 139 | 139 | 0.00 | 0.2474 |
| | Racemate | 3 | 138 | 141 | 2.13 | |
| Stupor | Levo- | 0 | 139 | 139 | 0.00 | 1.0000 |
| | Racemate | 1 | 140 | 141 | 0.71 | |
| Loss of appetite | Levo- | 0 | 139 | 139 | 0.00 | 1.0000 |
| | Racemate | 1 | 140 | 141 | 0.71 | |
| Stomach | Levo- | 0 | 139 | 139 | 0.00^{#} | 0.0145 |
| discomfort | Racemate | 7 | 134 | 141 | 4.96 | |
| Constipation | Levo- | 1 | 138 | 139 | 0.72 | 0.4964 |
| | Racemate | 0 | 141 | 141 | 0.00 | |
| Dry mouth | Levo- | 0 | 139 | 139 | 0.00 | 1.0000 |
| | Racemate | 1 | 140 | 141 | 0.71 | |
| Rash | Levo- | 0 | 139 | 139 | 0.00 | 0.4982 |
| | Racemate | 2 | 139 | 141 | 1.42 | |
| Pruritus | Levo- | 0 | 139 | 139 | 0.00 | 1.0000 |
| | Racemate | 1 | 140 | 141 | 0.71 | |
| Headache | Levo- | 0 | 139 | 139 | 0.00 | 1.0000 |
| | Racemate | 1 | 140 | 141 | 0.71 | |

^{#}P < 0.05, ^{##}P < 0.01, levo-ornidazole comparing with racemate.
AE: Adverse event

Data from Departments of Obstetrics and Gynecology

The total incidence rate of adverse events among patients from departments of obstetrics and gynecology was 3.50% (5 out of 143 patients) for levo-ornidazole, and 19.72% (28 out of 142 patients) for racemate ornidazole with a p-value <0.01 (Table 20).

TABLE 20
Adverse events observed for oral Levo- and racemate ornidazole in phase II human
clinical trials conducted among patients from departments of obstetrics/gynecology

| Adverse Event | Treatment | # of Patients | # of Patients | Total | Incidence | |
|---|---|---|---|---|---|---|
| (AE) | Groups | with AE | w/o AE | Patients | (%) | p-Value |
| Hypersomnia | Levo- | 0 | 143 | 143 | 0.00^{##} | 0.0034 |
| | Racemate | 8 | 134 | 142 | 5.63 | |
| Vertigo | Levo- | 0 | 143 | 143 | 0.00^{##} | 0.0034 |
| | Racemate | 8 | 134 | 142 | 5.63 | |
| Bloating | Levo- | 0 | 143 | 143 | 0.00 | 0.1224 |
| | Racemate | 3 | 139 | 142 | 2.11 | |
| Abdominal pain | Levo- | 0 | 143 | 143 | 0.00 | 0.4982 |
| | Racemate | 1 | 141 | 142 | 0.70 | |
| Stomach ache | Levo- | 1 | 142 | 143 | 0.70 | 1.0000 |
| | Racemate | 0 | 142 | 142 | 0.00 | |
| Vomiting | Levo- | 0 | 143 | 143 | 0.00 | 0.4982 |
| | Racemate | 1 | 141 | 142 | 0.70 | |
| Nausea | Levo- | 1 | 142 | 143 | 0.70^{#} | 0.0361 |
| | Racemate | 7 | 135 | 142 | 4.93 | |
| Hypodynamia | Levo- | 0 | 143 | 143 | 0.00 | 0.2474 |
| | Racemate | 2 | 140 | 142 | 1.41 | |
| Chest tightness | Levo- | 0 | 143 | 143 | 0.00 | 0.2474 |
| | Racemate | 2 | 140 | 142 | 1.41 | |
| Appetite drop | Levo- | 0 | 143 | 143 | 0.00 | 0.2474 |
| | Racemate | 2 | 140 | 142 | 1.41 | |
| Stomach | Levo- | 1 | 142 | 143 | 0.70 | 0.2138 |
| discomfort | Racemate | 4 | 138 | 142 | 2.82 | |
| Diarrhea | Levo- | 0 | 143 | 143 | 0.00 | 0.4982 |
| | Racemate | 1 | 141 | 142 | 0.70 | |
| Bitter taste | Levo- | 0 | 143 | 143 | 0.00 | 0.4982 |
| | Racemate | 1 | 141 | 142 | 0.70 | |
| Dry mouth | Levo- | 0 | 143 | 143 | 0.00 | 0.4982 |
| | Racemate | 1 | 141 | 142 | 0.70 | |
| Breast tenderness | Levo- | 0 | 143 | 143 | 0.00 | 0.4982 |
| | Racemate | 1 | 141 | 142 | 0.70 | |
| Oral malodor | Levo- | 1 | 142 | 143 | 0.70 | 1.0000 |
| | Racemate | 0 | 142 | 142 | 0.00 | |
| Blurred vision | Levo- | 0 | 143 | 143 | 0.00 | 0.4982 |
| | Racemate | 1 | 141 | 142 | 0.70 | |
| Rash | Levo- | 1 | 142 | 143 | 0.70 | 1.0000 |
| | Racemate | 0 | 142 | 142 | 0.00 | |
| Dazzle | Levo- | 0 | 143 | 143 | 0.00 | 0.4982 |
| | Racemate | 1 | 141 | 142 | 0.70 | |
| Dizziness | Levo- | 0 | 143 | 143 | 0.00 | 0.0603 |
| | Racemate | 4 | 138 | 142 | 2.82 | |
| Headache | Levo- | 0 | 143 | 143 | 0.00 | 0.4982 |
| | Racemate | 1 | 141 | 142 | 0.70 | |

^{#}P < 0.05, ^{##}P < 0.01, levo-ornidazole comparing with racemate.
AE: Adverse event

Under conventional doses to treat infections during clinical trials, patients experienced significantly fewer adverse events in the levo-ornidazole group than in the racemate group. All the observed adverse events are related to inhibition of the central nervous system, which correlates with the data from the molecular, cell and in vivo animal studies.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the following claims. All references cited hereby are incorporated by reference in their entirety.

What is claimed is:

1. A method for treating a parasitic infection, comprising administering to a patient in need of treatment a therapeutically effective amount of L-enantiomer enriched ornidazole, or a pharmaceutically acceptable salt or solvate thereof.

2. The method of claim 1, wherein the L-enantiomer enriched ornidazole, or a pharmaceutically acceptable salt or solvate thereof, has an enantiomeric excess of at least 90.0%.

3. The method of claim 1, wherein the L-enantiomer enriched ornidazole, or a pharmaceutically acceptable salt or solvate thereof, has an enantiomeric excess of at least 95.0%.

4. The method of claim 1, wherein the L-enantiomer enriched ornidazole is substantially enantiomerically pure L-ornidazole, or a pharmaceutically acceptable salt or solvate thereof.

5. The method of claim 1, wherein the parasitic infection is trichomonas vaginalis infection or cecal amoeba infection.

6. A method for treating a parasitic infection in a patient, comprising administering to the patient a pharmaceutical composition comprising a therapeutically effective amount of L-enantiomer enriched ornidazole, or a pharmaceutically acceptable salt or solvate thereof, and a pharmaceutically acceptable carrier.

7. The method of claim 6, wherein said L-enantiomer enriched ornidazole, or a pharmaceutically acceptable salt or solvate thereof, has an enantiomeric excess of at least 90.0%.

8. The method of claim 6, wherein said L-enantiomer enriched ornidazole, or a pharmaceutically acceptable salt or solvate thereof, has an enantiomeric excess of at least 95.0%.

9. The method of claim 6, wherein said L-enantiomer enriched ornidazole is substantially enantiomerically pure L-ornidazole, or a pharmaceutically acceptable salt or solvate thereof.

10. The method of claim 6, wherein the pharmaceutical composition is a pharmaceutical dosage form selected from tablets, capsules, and injectable solutions.

11. The method of claim 10, wherein the pharmaceutical dosage form is a tablet comprising L-ornidazole, pregelatinized starch, sodium starch glycolate, and magnesium stearate.

12. The method of claim 11, wherein the tablet is further coated by a membrane of opadry.

13. The method of claim 10, wherein the pharmaceutical dosage form is a capsule comprising L-ornidazole, starch, and magnesium stearate.

14. The method of claim 10, wherein the pharmaceutical dosage form is an injectable solution comprising L-ornidazole and a pharmaceutically acceptable carrier selected from the group consisting of sodium chloride, glucose and propylene glycol.

15. The method of claim 10, wherein the pharmaceutical dosage form is an effervescent tablet comprising L-ornidazole, sodium bicarbonate, low-substituted hydroxypropyl cellulose, sodium lauryl sulfate, microcrystalline cellulose, tartaric acid, and polyethylene glycol.

16. The method of claim 6, wherein the parasitic infection is trichomonas vaginalis infection or cecal amoeba infection.

17. The method of claim 15, wherein the parasitic infection is trichomonas vaginalis infection, and the effervescent tablet is a vaginal effervescent tablet.

18. The method of claim 6, wherein the pharmaceutical composition is a pharmaceutical dosage form suitable for an oral, intravenous, or vaginal delivery system.

19. The method of claim 18, wherein the pharmaceutical composition is administered orally at a dosage in the range of about 10-40 mg/kg/day.

20. The method of claim 18, wherein the pharmaceutical composition is administered intravenously at a dosage in the range of about 5-40 mg/kg/day.

21. The method of claim 18, wherein the pharmaceutical composition is administered intravaginally at a dosage in the range of about 10-40 mg/kg/day.

22. A pharmaceutical composition for use in the treatment of parasitic infections, comprising L-enantiomer enriched ornidazole, or a pharmaceutically acceptable salt or solvate thereof, and a pharmaceutically acceptable carrier.
